SEQUENCE LISTING (12 sequences)

SEQ ID NO: 1 - protein, 452 aa, Solanum commersonii
MAAMNEVIVAMVPFPDYSHLNQLFVLARFIASHNIQVHFLSFVDVNRDLELRLQGASNIHFENLLRPSSPTDKDGRDGEPPILMFLEKLTKPINEICVQLSTKARKLVIIHDLIMSEQVTDLNTLLNVKSYMFHTGSTFSRYSSLKQTIPDIVDDDDDHVKLIKQMQDEFPLLEPHEKYCFQVELDDDPVMHYSGEIINSCREMEGKYPDLLAKAKGKPLWAFGPFHMLLESHSNSSSNITRHDQCLEFLDKQDVNSVIFVSFGSTTTLSQDQVNELALGLEQSNHKFIWVLRKGDNAEKLKEKDIKIELPEGFEERVEGRGIMVNWAPQLEILGHSSTGGFMSHCGWNSCIESISMGVPVATWPISYDQPFNAIFLTNLLKIGISVKSWSHRNELIRASTIEKSVKTLMGTIEGEEMRRRAVELSKQIKNSVRSGGRARKDMESFISHIIE

SEQ ID NO: 2 - protein, 453 aa, Solanum commersonii
MAAINEMIVAMVPFPDYSHLNNLFDLARFIASHNIQVHFLCFVDVNRDLKLRLQGSLGVSNIHFENLLRPSSPTDKDGRDGEPPILMFLEKLTKPINEICVQLSTKARKLVIIHDLIMSEQITDLNTLLNVKSYMFHTGSTFSRYSSLKQTIPDIVDDDDDHVKLIKQMQDEFPLLEPHEKYCFQMELDDDMRYSGEIINSYREMEGKYPDLLAKAKGKPLWAFGPFHMLLESHSNSSRNITRHDQCLEFLDKQDVNSVIFVSFGSTTTLSQKQVNELALGLEQSNHKFIWVLRKGDNAEKLKEKDIKIELPEGFEERVEGRGIMVNWAPQLEILGHSSTGGFMSHCGWNSCIESISMGVPVATWPISYDQPFNAIFLTNLLKIGISVKSWSHRNEVITASTIEKSVKTLMGTIEGEEMRRRAVELSKQIKNSVRSGGRARKDMESFISHIIE

SEQ ID NO: 3 - DNA, 1359 bp, Solanum commersonii
atggctgctatgaatgaagtgattgtggccatggtgccattccctgattacagccatctcaatcagctcttcgttcttgctcgtttcatcgcgtcccataatatacaagtacacttcctctcctttgttgatgtgaaccgagatttggaacttcgcctccaaggggcctcaaacatccatttcgaaaacctgttgagaccttcttctccaacagataaagatggtcgcgatggagagcctccgattctaatgtttctggaaaaactcacgaagcccattaatgaaatatgtgttcaactctctactaaggcaagaaaattagtcataattcatgaccttataatgagtgaacaagtaacggatctaaatacattgttgaatgtcaagtcttatatgttccacaccggatcgacatttagcagatattcttccctcaaacaaactattcctgacatagttgacgatgacgatgatcatgtaaagttgatcaagcaaatgcaggatgagtttccattgttggagccacatgagaaatattgttttcaagtggagcttgatgatgatccggttatgcattactccggagaaatcataaactcgtgtagagaaatggaaggtaagtacccagatttacttgccaaagcaaaaggcaagccgttatgggcttttggtccatttcatatgttgctagaatcacactctaattcgtctagcaacatcactcgtcatgatcaatgcctagaatttctcgacaaacaagatgttaactcagtgatattcgtctcgtttggatcaaccactacattatcacaagaccaagtcaatgagcttgctttgggtttagaacaaagcaaccataaatttatttgggtactaagaaaaggagataacgcggagaaacttaaagagaaagacataaagattgaattacccgaagggtttgaagagagagtggaaggaagaggaataatggttaattgggcaccacagttggaaatcttggggcattcatctacgggtgggtttatgagtcactgtggatggaattcttgtatcgagagcattagcatgggagtacctgtagcaacttggccaatcagttacgatcagccatttaacgctatatttttaaccaatttgttgaagattggaatctctgtcaagagttggtctcaccggaatgaattgataagagcatcaaccattgagaaatctgtcaagacattgatgggtacgatagaaggggaagagatgaggcgaagggcagtggaattgagcaaacagatcaaaaactctgttagaagcggaggacgtgcacgcaaggatatggaatctttcatatcacatattattgaataa

SEQ ID NO: 4 - DNA, 1362 bp, Solanum commersonii
atggctgctatcaatgaaatgattgtggccatggtgccattccctgattacagccatctcaataatctcttcgatcttgctcgtttcatcgcgtcccataatatacaagtacacttcctctgctttgttgatgtgaaccgagatttgaaacttcgcctccaaggtagtctcggggtctcaaacatccatttcgaaaacctgttgagaccttcttctccaacagataaagatggtcgcgatggagagcctccgattctaatgtttctggaaaaactcacgaagcccattaatgaaatatgtgttcaactatctactaaggcaagaaaattagtcataattcatgaccttataatgagtgaacaaataacggatctaaatacattgttgaatgtcaagtcttatatgttccacaccggatcgacatttagcagatattcttccctcaaacaaactattcctgacatagttgacgatgacgatgatcatgtaaaattgatcaagcaaatgcaggatgagtttccattgttggagccacatgagaaatattgttttcaaatggagcttgatgatgatatgcgttactccggagaaatcataaactcgtatagagaaatggaaggtaagtacccagatttacttgccaaagcaaaaggcaagccgttatgggcttttggtccatttcatatgttgctagaatcacactctaattcgtctagaaacatcactcgtcatgatcaatgcctagaatttctcgacaaacaagatgttaactcagtgatattcgtctcgtttggatcaaccactacattatcacaaaagcaagtcaatgagcttgctttgggtttagaacaaagcaaccataaatttatttgggtactaagaaaaggagataacgcggagaaacttaaagagaaagacataaagattgaattacccgaagggtttgaagagagagtggaaggaagaggaataatggttaattgggcaccacagttggaaatcttggggcattcatctacgggtgggtttatgagtcactgtggatggaattcttgtatcgagagcattagcatgggagtacctgtagcaacttggccaatcagttacgatcagccatttaacgctatatttttaaccaatttgttgaagattggaatctctgtcaagagttggtctcaccggaatgaagtgataacagcatcaaccattgagaaatctgtcaagacattgatgggtacgatagaaggggaagagatgaggcgaagggcagtggaattgagcaaacagatcaaaaactctgttagaagcggaggacgtgcacgcaaggatatggaatctttcatatcacatattattgaataa

SEQ ID NO: 5 - DNA, 20 bp, Artificial Sequence
  E2 forward
gtttctcagcaacaggcaaa

SEQ ID NO: 6 - DNA, 21 bp, Artificial Sequence
  E2 reverse
tgtctctggtatggagcctgt

SEQ ID NO: 7 - DNA, 19 bp, Artificial Sequence
  E3 forward
gaccctctggcaagcaaat

SEQ ID NO: 8 - DNA, 20 bp, Artificial Sequence
  E3 reverse
tcatcatcgttcatggcatt

SEQ ID NO: 9 - DNA, 1320 bp, Solanum commersonii
atgggaaagttgagaattgtaatgttgccatacttagcttatgggcacatcacacctttccttgaactagccaagaaactctcaaatagaggtttctccattgacatatgttcttctcccattaatcttagcttcatcaaggaaaaaatcccagaaaaatactcttcctcaattcatctaattgaacttcacttaccaaacttacctgaacttcctcctcatcaccacacaactaatggccttccaagtcatcttaaacaaactcttttcaaagcacttaagatgaccaaaccacagtttcaccaaatcttgaatgataaaaaacctgattttttgatatatgatatcatgttactatggtcaggtgtagttgcatcttcactcaacatcccatcattaaatttctacactgtaaacgcagctatttttagttattttttccatttttattacaatccaggagaggaattccctttcccagctctgtatatgagggattatgagcaagccaaaatgacacatgaagttgttgatgacgcggaagttgaagttgacaaagacaatgttactgaatcagacaaatttgtgcttgttcatagctctaaatcaatagatgggaagtacatggactatctttgtggaacaggacaagcaaaagttgtaccaattggaacacaatctcctgaagatgatgttggtgatcttgatattgaactcctcaaatggcttgagaagaaaacagatcattcaactgtttacgtctcatttgggagcgagtatttcttaagcaaagaagaaatggaagaggtagcttatggattagagcttagcggtgctgatttcatatgggttgttaggtatcaaaaaggggaacaagtagaacttcctcaaggttttaaagaaagaattggagagagaggatggataattgaaggatgggctccgcaacaaagaattctaaagcattcaagtattggtggatttgtaacgcattgtggttggaattcgacactagagagcatagagtttggtgttccaatcatagctatgcctatgctttatgatcagcctttgaatgctagattgatggtggaaaatggagtagctgttgaggtcccaagagatgaaaatgggaatcttgatagacttaatatagctgagaaaattaaacatgtgattagtgacgaaacaggggagaatttgaggaagaagatgaataatcttggtgagaatgtgagatctcaaagagaagacgagatggatggagttgttgaggttatacaactcttgattgatgagaagaattcccagaaatcataa

SEQ ID NO: 10 - protein, 439 aa, Solanum commersonii
MGKLRIVMLPYLAYGHITPFLELAKKLSNRGFSIDICSSPINLSFIKEKIPEKYSSSIHLIELHLPNLPELPPHHHTTNGLPSHLKQTLFKALKMTKPQFHQILNDKKPDFLIYDIMLLWSGVVASSLNIPSLNFYTVNAAIFSYFFHFYYNPGEEFPFPALYMRDYEQAKMTHEVVDDAEVEVDKDNVTESDKFVLVHSSKSIDGKYMDYLCGTGQAKVVPIGTQSPEDDVGDLDIELLKWLEKKTDHSTVYVSFGSEYFLSKEEMEEVAYGLELSGADFIWVVRYQKGEQVELPQGFKERIGERGWIIEGWAPQQRILKHSSIGGFVTHCGWNSTLESIEFGVPIIAMPMLYDQPLNARLMVENGVAVEVPRDENGNLDRLNIAEKIKHVISDETGENLRKKMNNLGENVRSQREDEMDGVVEVIQLLIDEKNSQKS

SEQ ID NO: 11 - DNA, 1395 bp, Solanum commersonii
atgcaattagcatcttcacagatggataacaaagatcatgatgtagctgtagtgatagctccattacatcttcaaagccatctccgacaacttctccattttgcatgccgaatttcctcctacggtgtaccggtctactatcttggctcagccacggctaaccatcaggctagactccactccactaccttaaattcttcggacatagccaaaatccactttcatgacctcgaaattcctactcaagatccacctccgaatgtcccaatgcatatttggaatgcttctatgcaaacacgtgaacccattgcttctttctttcgagatatctcctctaaggcaagacgaattgttgttgttcacgatcttttaatgtcgtataatgttcaggatatttcttcatatcccaatggagaatcctatgtatttcattgcctacctattttcgatatgtattgtagtcactatgccccagcagcagggttgcctattccacttgaagaagaacttcttaaaaagttaccctctaatgatgattgttacaatcctgaggatgtcgagcaccatgcaaagtatctaaatcattgcatgggaaagaatgctggtgatatctttaatacaagtcaaataattgatggtactgcttttgttgattccatggcacatcttgcaaccatacaaaacaaaatgttatgggcactcggaccaattctccttacacaagatcatgataagatccaaatcaaacacctatgtttggattggcttaacaaacaacctccgaaatcagttctttatgtatcttttggaacatctacttcattttcaactcatcaaatcaaggagcttgcaattggtttagagctaagcaaacaaaagttcatatgggtatttagaaatgctgatgaaggagatcccgttactaataaatgcgaaaataatagattaggaccacttgagttgcctcaaggatttgaagaaagggtaaaaggtgttggtttagtggtgaaagaatgggcaccccaacaagaaattttggctcattcttctactggtgggttcatgagtcattgtggatggacttcatgcttagaaagcattattgaaggtgtgccaatagcggcttggcctatccaatttgaccaaccaaaaaatggttttttagtcgcagaagtactaaaaatagggctccatgttagggagtgggagcaacggaatgagttagtgactgcatccaccattcagaatatagtgagcaaattaatggcatcagaagacggtgatatgattaggaaaagagcacaagaactaagcaaggccgtaagacggtccacggaggaaggtggtgtttctcgaatggagttggagtctttcgtggcgcatatcacaagataa

SEQ ID NO: 12 - protein, 464 aa, Solanum commersonii
MQLASSQMDNKDHDVAVVIAPLHLQSHLRQLLHFACRISSYGVPVYYLGSATANHQARLHSTTLNSSDIAKIHFHDLEIPTQDPPPNVPMHIWNASMQTREPIASFFRDISSKARRIVVVHDLLMSYNVQDISSYPNGESYVFHCLPIFDMYCSHYAPAAGLPIPLEEELLKKLPSNDDCYNPEDVEHHAKYLNHCMGKNAGDIFNTSQIIDGTAFVDSMAHLATIQNKMLWALGPILLTQDHDKIQIKHLCLDWLNKQPPKSVLYVSFGTSTSFSTHQIKELAIGLELSKQKFIWVFRNADEGDPVTNKCENNRLGPLELPQGFEERVKGVGLVVKEWAPQQEILAHSSTGGFMSHCGWTSCLESIIEGVPIAAWPIQFDQPKNGFLVAEVLKIGLHVREWEQRNELVTASTIQNIVSKLMASEDGDMIRKRAQELSKAVRRSTEEGGVSRMELESFVAHITR

(19) United States
(12) Patent Application Publication
(10) Pub. No.: US 2023/0002782 A1
(43) Pub. Date: Jan. 5, 2023

(54) PEST AND PATHOGEN RESISTANCE IN PLANTS

(71) Applicant: J.R. Simplot Company, Boise, ID (US)

(72) Inventors: Craig RICHAEL, Meridian, ID (US); Vivianne Gertruda Antonia Anna VLEESHOUWERS, Wageningen (NL); Richard Gerardus Franciscus VISSER, Wageningen (NL); Pieter Jacobus WOLTERS, Wageningen (NL)

(21) Appl. No.: 17/842,515

(22) Filed: Jun. 16, 2022

Related U.S. Application Data
(60) Provisional application No. 63/211,154, filed on Jun. 16, 2021.

(51) Int. Cl. C12N 15/82

(52) U.S. Cl. CPC C12N 15/8282; C12N 15/8218

(57) ABSTRACT

The present disclosure relates to methods of generating plants resistant to pests and pathogens, and plants produced therefrom. The disclosure further relates to methods of identifying plants having a resistant gene(s). The disclosure further relates to compositions for controlling plant pests and pathogens.

### CROSS-REFERENCE TO RELATED APPLICATIONS

This application claims the benefit of priority to U.S. Provisional Application No. 63/211,154, filed on Jun. 16, 2021, which is hereby incorporated by reference in its entirety for all purposes.

### FIELD

The present disclosure relates to methods and compositions for controlling or providing resistance to pests and pathogens, such as fungal and fungal-like pathogens. Also provided are methods for transforming plants with the disclosed sequences, detecting said sequences, and plants produced therefrom.

### DESCRIPTION OF THE TEXT FILE SUBMITTED ELECTRONICALLY

The contents of the text file submitted electronically herewith are incorporated herein by reference in their entirety: A computer readable format copy of the Sequence Listing filename: JRSI_084_01US_SeqList_ST25.txt, date recorded, Jun. 15, 2022, file size ≈24,155 bytes.

### BACKGROUND

Pests such as insects, arachnids, gastropods, fungal pathogens, mites, and nematodes are detrimental, causing economic loss of plant crops. Methods for controlling pests and pathogens often include spraying plants with harsh chemicals. Typical fungicides used include compounds of heavy metals such as copper and arsenic, as well as organosulfur and organochlorine compounds. These compounds are often not satisfactory because of their potential for accumulating in the environment to levels considered to be unsafe, in particular contaminating natural resources such as drinking water and polluting the soil. Further, fungicides and other pesticides have strong physiological effects on the plants, may produce residues that are toxic to food crops, can have high animal toxicity, and are potentially hazardous to workers using them.

Therefore, there is a need to identify resistant genes and compounds and develop compositions and methods for controlling pests and pathogens.

### SUMMARY OF THE DISCLOSURE

The following embodiments and aspects thereof are described in conjunction with systems, tools and methods which are meant to be exemplary, not limiting in scope. In various embodiments, one or more of the above-described problems have been reduced or eliminated, while other embodiments are directed to other improvements.

The present disclosure provides a method for conferring resistance or tolerance to a pest or pathogen in a Solanum spp. plant, plant part, or plant cell, comprising at least one of: introducing a nucleic acid into a Solanum spp. plant, plant part, or plant cell, wherein the nucleic acid encodes an amino acid sequence at least 90% identical to SEQ ID NO: 1, and introducing a nucleic acid into a Solanum spp. plant, plant part, or plant cell, wherein the nucleic acid encodes an amino acid sequence at least 90% identical to SEQ ID NO: 2. In some embodiments, the method further comprises introducing a nucleic acid encoding an amino acid sequence at least 90% identical to SEQ ID NO: 10. In some embodiments, the method further comprises introducing a nucleic acid encoding an amino acid sequence at least 90% identical to SEQ ID NO: 12. In some embodiments, the present disclosure provides plants produced from the disclosed methods.

The disclosure further provides a method for conferring resistance to a fungal pathogen in a Solanum tuberosum plant, plant part, or plant cell, comprising: (a) introducing a nucleic acid into a S. tuberosum plant, wherein the nucleic acid encodes an amino acid sequence at least 90% identical to SEQ ID NO: 1, and/or introducing a nucleic acid into a Solanum spp. plant, plant part, or plant cell, wherein the nucleic acid encodes an amino acid sequence at least 90% identical to SEQ ID NO: 2; and (b) introducing a nucleic acid into the Solanum spp. plant, plant part, or plant cell, wherein the nucleic acid encodes at least one functional homolog of a glycoalkaloid metabolism (GAME) polypeptide that is a glycotransferase; wherein expression of the nucleic acids in (a) and (b) produce tetraose glycoalkaloids, and wherein the tetraose glycoalkaloids confer resistance to a fungal pathogen. In some embodiments, the present disclosure provides plants produced from the disclosed methods.

The disclosure further provides compositions comprising an effective amount of isolated tetraose glycoalkaloids and an adjuvant.

The disclosure further provides a method for controlling fungal and fungal-like pathogens on a plant, comprising applying an effective amount of isolated tetraose glycoalkaloids.

The disclosure further provides a plant transformation vector designated pSIMScGTR1 comprising the nucleotide sequence set forth in SEQ ID NO: 3, and wherein the plant transformation vector was deposited under ATCC Accession No. PTA-______.

The disclosure further provides a plant transformation vector designated pSIMScGTR2 comprising the nucleotide sequence set forth in SEQ ID NO: 4, and wherein the plant transformation vector was deposited under ATCC Accession No. PTA-______.

The disclosure further provides a plant transformation vector designated pSIML2GGA1 comprising the nucleotide sequences set forth in SEQ ID NOs: 3 and 9, and wherein the plant transformation vector was deposited under ATCC Accession No. PTA-______.

The disclosure further provides a plant transformation vector designated pSIML2GGA2 comprising the nucleotide sequences set forth in SEQ ID NOs: 4 and 9, and wherein the plant transformation vector was deposited under ATCC Accession No. PTA-______.

The disclosure further provides a plant transformation vector designated pSIML2GGA3 comprising the nucleotide sequences set forth in SEQ ID NOs: 3, 4, and 9, and wherein the plant transformation vector was deposited under ATCC Accession No. PTA-______.

The disclosure further provides plant transformation vectors comprising at least one of: a nucleotide sequence encoding an amino acid sequence at least 90% identical to SEQ ID NO: 1, a nucleotide sequence encoding an amino acid sequence at least 90% identical to SEQ ID NO: 2; a nucleotide sequence encoding an amino acid sequence at least 90% identical to SEQ ID NO: 10; and a nucleotide sequence encoding an amino acid sequence at least 90% identical to SEQ ID NO: 12, and plants comprising said vectors.

The disclosure further provides Solanum tuberosum plants, or parts thereof, comprising a nucleic acid encoding an amino acid sequence at least 90% identical to SEQ ID NO: 1.

The disclosure further provides Solanum tuberosum plants, or parts thereof, comprising a nucleic acid encoding an amino acid sequence at least 90% identical to SEQ ID NO: 2.

The disclosure further provides Solanum tuberosum plants, or parts thereof, comprising a nucleic acid encoding an amino acid sequence at least 90% identical to SEQ ID NO: 10.

The disclosure further provides Solanum tuberosum plants, or parts thereof, comprising a nucleic acid encoding an amino acid sequence at least 90% identical to SEQ ID NO: 12.

The disclosure further provides a sequence fragment of at least 15 consecutive nucleotides of the ScGTR1 gene described by SEQ ID NO: 3 or its complementary sequence.

The disclosure further provides a sequence fragment of at least 15 consecutive nucleotides of the ScGTR2 gene described by SEQ ID NO: 4 or its complementary sequence.

The disclosure further provides a sequence fragment of at least 15 consecutive nucleotides of the ScGAME18 gene described by SEQ ID NO: 9 or its complementary sequence.

The disclosure further provides a sequence fragment of at least 15 consecutive nucleotides of the ScGAME17 gene described by SEQ ID NO: 11 or its complementary sequence.

In addition to the exemplary aspects and embodiments described above, further aspects and embodiments will become apparent by the following descriptions.

### BRIEF DESCRIPTION OF THE DRAWINGS

FIG. 1A shows BSA-RNAseq results. SNPs were identified by aligning RNAseq reads from the parents of AJW12 and pools of resistant/susceptible progeny to the potato DM v4.03 genome. (Potato Genome Sequencing Consortium 2011, Genome sequence and analysis of the tuber crop potato. Nature 475: 189-195; Sharma, S. K., et al., 2013, Construction of Reference Chromosome-Scale Pseudomolecules for Potato: Integrating the Potato Genome with Genetic and Physical Maps. G3: Genes|Penomes|Penetics 3: 2031-2047). They were filtered to identify candidate SNPs linked to the S. commersonii resistance. The number of remaining SNPs in windows of 100 Kb were plotted over the 12 chromosome of the potato genome. Almost all filtered SNPs map to chromosome 12, indicating that the S. commersonii resistance gene is located on that chromosome.

FIG. 1B shows the mapping of the S. commersonii resistance gene. Filtered SNPs were used as markers in a High-Resolution Melting (HRM) analysis of the parents of AJW12 (CGN18024_1×CGN18024_3), as well as 28 progeny plants. Green depicts markers in coupling phase with resistance and orange those in repulsion phase. Four recombinants were found, which limit the genetic window for the resistance between marker B1 and B3, corresponding to a region of approximately 3 Mb based on the homologous chromosome from the DM v4.03 genome.

FIG. 1C shows an overview of the resistance region in the susceptible/resistant haplotype of CGN18024_1. The resistance region delimited by markers E2 (SEQ ID NO: 5 and SEQ ID NO: 6) and E3 (SEQ ID NO: 7 and SEQ ID NO: 8) from the resistant haplotype (UTG_R) and the corresponding region in the susceptible haplotype (UTG_S) are displayed. Positions of markers used in fine-mapping are indicated above and below UTG_S and UTG R respectively. The major differences between UTG_R and UTG_S are an insertion of ˜3.7 kb (white) in UTG_S and a duplication of about 9.9 kb (purple) in UTG R, resulting in a fine-mapped resistance region of ˜26 kb on UTG R (between markers 797K and 817K). The corresponding region on UTG_S is ˜20 K kb.

FIG. 2A-2C shows an overview of the fine-mapped resistance region on UTG_R. FIG. 2A depicts the fine mapped region (between markers 797K and 817K) is displayed at the top and black bars indicate whether DNBseq reads from CGN18024_1 and CGN18024_3 map to the region. Reads from CGN180249_1 cover the complete resistance region, whereas the region is only partly covered by reads from CGN18024_3. FIG. 2B shows the location of transcripts predicted by Augustus software (available on the world wide web at pubmed.ncbi.nlm.nih.gov/14534192/). FIG. 2C shows the RNAseq coverage in the resistance region. Predicted transcripts g104865 and g104867 are expressed in CGN18024_1 (the resistant parent of population AJW12) as well as the bulk of resistant progeny, but not in CGN18024_3 (the susceptible parent of population AJW12) or the bulk of susceptible progeny.

FIG. 3A-3D shows early blight disease test on S. commersonii (FIG. 3A & FIG. 3B) and S. tuberosum cv Atlantic transiently expressing ScGTR1 (‘R1’) ScGTR2 (‘R2’), the corresponding non-functional allele from the susceptible haplotype (‘S2’) and empty vector control (‘-’). Average lesion sizes (FIG. 3A and FIG. 3C) and representative pictures of CGN18024_3 (FIG. 3B) and Atlantic (FIG. 3D) are shown. CGN18024_1 is resistant to A. solani, independent of the treatment, whereas CGN18024_3 is susceptible, except when ScGTR1 and ScGTR2 are transiently expressed. No effect of transiently expressing ScGTR1 and ScGTR2 is seen in S. tuberosum cv Atlantic or Bintje variety.

FIG. 4A shows photographs of plates inoculated with A. solani having either 5% autoclaved leaf extract from CGN18024_1 (resistant strain) to PDA medium (left), or 5% autoclaved leaf extract from CGN18024_3 (susceptible strain) to a PDA plate (right). The picture is taken 11 days after placing a PDA plug containing mycelium of A. solani at the center of each plate.

FIG. 4B shows photographs of plates inoculated with A. solani and having fungal contamination. The plate on the left had 5% autoclaved leaf extract from CGN18024_1 (resistant strain) added to the PDA medium, whereas the plate on the right had 5% autoclaved leaf extract from CGN18024_3 (susceptible strain) added to the a PDA medium. The picture is taken 4 days after placing a PDA plug containing mycelium of A. solani at the center of each plate.

FIG. 4C is a line graph plotting the fungal colony diameter (mm) of A. solani over the course of 7 days for PDA media plates containing different concentrations of extracts from CGN18024_1 and CGN18024_3.

FIG. 4D is a line graph plotting the fungal colony diameter (mm) of Botrytis cinerea over the course of 4 days for PDA media plates containing different concentrations of extracts from CGN18024_1 and CGN18024_3.

FIG. 5 shows a bar graph of glycoalkaloids identified in resistant and susceptible S. commersonii. The analysis was performed on leaves from CGN18024_1 (R, resistant to A. solani), CGN18024_3 (S, susceptible to A. solani) and resistant/susceptible CGN18024_3 transformed with ScGTR1 or ScGTR2.

FIG. 6A-6C shows a proposed biosynthesis of glycoalkaloids in tomato (Solanum lycopersicum) (A) compared with what is known in wild varieties of potato (Solanum commersonii) (B) and cultivated potato (Solanum tuberosum) (C).

FIG. 7 shows a phylogenetic tree of glycosyltransferases from S. lycopersicum (SI), S. tubersum (St), and S. commersonii (Sc).

FIG. 8A-8C provide schematic representations of the transformation vectors pSIML2GGA1 (FIG. 8A), which comprises ScGTR1 (SEQ ID NO: 3) and ScGAME18 (SEQ ID NO: 9), pSIML2GGA2 (FIG. 8B). which comprises ScGTR2 (SEQ ID NO: 4) and ScGAME18 (SEQ ID NO: 9), and pSIML2GGA3 (FIG. 8C), which comprises ScGTR1 (SEQ ID NO: 3), ScGTR2 (SEQ ID NO: 4) and ScGAME18 (SEQ ID NO: 9).

### DETAILED DESCRIPTION

### Definitions

In the description and tables which follow, a number of terms are used. In order to provide a clear and consistent understanding of the specification and claims, including the scope to be given such terms, the following definitions are provided.

The term “a” or “an” refers to one or more of that entity; for example, “a primer” refers to one or more primers or at least one primer. As such, the terms “a” (or “an”), “one or more” and “at least one” are used interchangeably herein. In addition, reference to “an element” by the indefinite article “a” or “an” does not exclude the possibility that more than one of the elements is present, unless the context clearly requires that there is one and only one of the elements.

Artificially manipulated. as used herein, “artificially manipulated” means to move, arrange, operate or control by the hands or by mechanical means or recombinant means, such as by genetic engineering techniques, a plant or plant cell, so as to produce a plant or plant cell that has a different biological, biochemical, morphological, or physiological phenotype and/or genotype in comparison to unmanipulated, naturally-occurring counterpart.

Backbone. Nucleic acid sequence of a vector that excludes the DNA insert sequence intended for transfer.

Broad-spectrum. A “broad spectrum compound” is a compound having broad spectrum activity, which is effective against a range of organisms. For example, a broad-spectrum fungicide is effectively active against multiple pathogens (i.e. fungi).

Degenerate primer. A “degenerate primer” is an oligonucleotide that contains sufficient nucleotide variations that it can accommodate base mismatches when hybridized to sequences of similar, but not exact, homology.

Early blight. Early blight is a fungal disease in plants caused by species of the Alternaria genus. It is a common disease in plants of the Solanum genus, for example, tomatoes and potatoes.

Event. Event refers to the unique DNA recombination event that took place in one plant cell, which was then used to generate entire transgenic plants. Plant cells are transformed with a vector carrying a DNA insert of interest. Transformed cells are regenerated into transgenic plants, and each resulting transgenic plant represents a unique event. Different events possess differences in the number of copies of DNA insert in the cell genome, the arrangement of the DNA insert copies and/or the DNA insert location in the genome.

Foreign. “Foreign” with respect to a nucleic acid is non-native nucleic acid.

Functional homolog. In this disclosure, “functional homolog” refers to a protein or a gene (DNA sequence) encoding a protein that shares the same or similar functions in a biological, biochemical, catalytic or metabolic process. The “functional homolog” may be an actual homolog by virtue of descent from a common ancestor, or it may be unrelated (for example, an analogous sequence or protein arisen from convergent evolution).

Fungal-like. As used herein, “fungal-like” refers to those pathogens which respond to fungicides, including, but not limited to, for example, Plasmopara viticola and P. infestans.

Single locus Converted (Conversion). Single locus converted (conversion) plant refers to plants wherein essentially all of the desired morphological and physiological characteristics of a variety are recovered in addition to the one or more loci transferred into the variety via, for example, the backcrossing technique, via genetic engineering or via mutation.

Genetic rearrangement. Refers to the re-association of genetic elements that can occur spontaneously in vivo as well as in vitro which introduce a new organization of genetic material. For instance, the splicing together of polynucleotides at different chromosomal loci, can occur spontaneously in vivo during both plant development and sexual recombination. Accordingly, recombination of genetic elements by non-natural genetic modification techniques in vitro is akin to recombination events that also can occur through sexual recombination in vivo.

Isolated. “Isolated” refers to any nucleic acid or compound that is physically separated from its normal, native environment. The isolated material may be maintained in a suitable solution containing, for instance, a solvent, a buffer, an ion, or other component, and may be in purified, or unpurified, form. Isolated may also refer to nucleic acid that has been isolated from an organism and is maintained, for example, in a plasmid.

Late blight. A potato disease caused by the oomycete Phytophthora infestans and also known as ‘potato blight’ that can infect and destroy the leaves, stems, fruits, and tubers of potato plants.

Marketable Yield. Marketable yield is the weight of all tubers harvested that are between 2 and 4 inches in diameter. Marketable yield is measured in cwt (hundred weight) where cwt=100 pounds.

Native. A “native” genetic element refers to a nucleic acid that naturally exists in, originates from, or belongs to the genome of a plant that is to be transformed.

Non-natural nucleotide junction. “Non-natural nucleotide junction” or “non-naturally occurring nucleotide junction” refers to a sequence of nucleotides that do not occur in nature. Rather, these sequences are formed via a genetic transformation event. For example, the genetic transformation events described herein may be created with expression cassettes that contain no non-native potato DNA. Thus, these non-natural nucleotide junctions are composed of potato nucleotides, but these nucleotides are in a genetic arrangement that does not occur in nature, but which results from the manipulation of man that occurs during the genetic transformation of the potato.

Operably linked. Combining two or more molecules in such a fashion that in combination they function properly in a plant cell. For instance, a promoter is operably linked to a structural gene when the promoter controls transcription of the structural gene.

Plant. As used herein, the term “plant” includes a monocot or a dicot. The word “plant,” as used herein, also encompasses plant cells, seed, plant progeny, propagule whether generated sexually or asexually, and descendants of any of these, such as cuttings or seed. Plant cells include suspension cultures, callus, embryos, meristematic regions, callus tissue, leaves, roots, shoots, gametophytes, sporophytes, pollen, seeds and microspores.

Plant Parts. As used herein, the term “plant parts” (or a potato plant, or a part thereof) includes but is not limited to protoplast, leaf, stem, root, root tip, anther, pistil, seed, embryo, pollen, ovule, cotyledon, hypocotyl, flower, tuber, eye, tissue, petiole, cell, meristematic cell, and the like.

Progeny. As used herein, includes an Fi potato plant produced from the cross of two potato plants where at least one plant includes the selected event and progeny further includes, but is not limited to, subsequent F₂, F₃, F₄, F₅, F₆, F₇, F₈, F₉, generations.

Quantitative Trait Loci (QTL). Quantitative trait loci (QTL) refer to genetic loci that control to some degree numerically representable traits that are usually continuously distributed.

Recombinant. As used herein, broadly describes various technologies whereby genes can be cloned, DNA can be sequenced, and protein products can be produced. As used herein, the term also describes proteins that have been produced following the transfer of genes into the cells of plant host systems.

Regeneration. Regeneration refers to the development of a plant from tissue culture.

Regulatory sequences. Refers to those sequences which are standard and known to those in the art that may be included in the expression vectors to increase and/or maximize transcription of a gene of interest or translation of the resulting RNA in a plant system. These include, but are not limited to, promoters, peptide export signal sequences, introns, polyadenylation, and transcription termination sites.

Selectable marker. A “selectable marker” is used to identify transformation events.

Specific gravity. As used herein, “specific gravity” is an expression of density and is a measurement of potato quality. There is a high correlation between the specific gravity of the tuber and the starch content and percentage of dry matter or total solids. A higher specific gravity contributes to higher recovery rate and better quality of the processed product.

Sequence identity. “sequence identity” or “identity” in the context of two nucleic acid or polypeptide sequences includes reference to the number of residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity.” Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988). The comparison of sequences and determination of percent identity between two sequences can be accomplished using a mathematical algorithm. An example of a local alignment algorithm utilized for the comparison of sequences is the NCBI Basic Local Alignment Search Tool (BLAST®) (Altschul et al. 1990 J. Mol. Biol. 215: 403-10), which is available from several sources, including the National Center for Biotechnology Information (NCBI, Bethesda, Md.) and on the Internet, for use in connection with the sequence analysis programs blastp, blastn, blastx, tblastn and tblastx. It can be accessed on the internet via the National Library of Medicine (NLM)'s world-wide-web URL. A description of how to determine sequence identity using this program is available at the NLM's website on BLAST tutorial. Another example of a mathematical algorithm utilized for the global comparison of sequences is the Clustal W and Clustal X (Larkin et al. 2007 Bioinformatics, 23, 2947-294, Clustal W and Clustal X version 2.0) as well as Clustal omega. Unless otherwise stated, references to sequence identity used herein refer to the NCBI Basic Local Alignment Search Tool (BLAST®).

Total Yield. Total yield refers to the total weight of all harvested tubers.

Transformation of plant cells. A process by which DNA is integrated into the genome of a plant cell. The integration may be transient or stable. “Stably” refers to the permanent, or non-transient retention and/or expression of a polynucleotide in and by a cell genome. Thus, a stably integrated polynucleotide is one that is a fixture within a transformed cell genome and can be replicated and propagated through successive progeny of the cell or resultant transformed plant. Transformation may occur under natural or artificial conditions using various methods well known in the art. Transformation may rely on any known method for the insertion of nucleic acid sequences into a prokaryotic or eukaryotic host cell, including Agrobacterium-mediated transformation protocols, viral infection, whiskers, electroporation, heat shock, lipofection, polyethylene glycol treatment, micro-injection, and particle bombardment.

Transgenic plant. A genetically modified plant which contains at least one transgene.

Variant. A “variant,” as used herein, is understood to mean a nucleotide or amino acid sequence that deviates from the standard, or given, nucleotide or amino acid sequence of a particular gene or protein. The terms, “isoform,” “isotype,” and “analog” also refer to “variant” forms of a nucleotide or an amino acid sequence. An amino acid sequence that is altered by the addition, removal or substitution of one or more amino acids, or a change in nucleotide sequence, may be considered a “variant” sequence. The variant may have “conservative” changes, wherein a substituted amino acid has similar structural or chemical properties, e.g., replacement of leucine with isoleucine. A variant may have “nonconservative” changes, e.g., replacement of a glycine with a tryptophan. Analogous minor variations may also include amino acid deletions or insertions, or both. Guidance in determining which amino acid residues may be substituted, inserted, or deleted may be found using computer programs well known in the art such as Vector NTI Suite (InforMax, Md.) software, CLC Genomics Workbench (Germantown, Md.) or EMBL-EBI online software.

Vine Maturity. Vine maturity refers to a plant's ability to continue to utilize carbohydrates and photosynthesize. Vine maturity is scored on a scale of 1 to 5 where 1 =dead vines and 5=vines green, still flowering.

Solanum tuberosum. As used herein, “S. tuberosum” includes S. tuberosum subsp. tuberosum, S. tuberosum Group Ajanhuiri, S. tuberosum Group Andigenum, S. tuberosum Group Chaucha, S. tuberosum Group Chilotanum, S. tuberosum Group Curtilobum, S. tuberosum Group Juzepczukii, S. tuberosum Group Phureja, and S. tuberosum Group Stenotomum.

### Overview

The present disclosure relates to methods of generating plants resistant to pests and pathogens, and plants produced therefrom. The disclosure further relates to methods of identifying plants having a resistant gene(s). The disclosure further relates to compositions for controlling plant pests and pathogens.

### The pathway to Resistance

Early blight is a fungal disease common in potato and tomato plants. It is caused primarily by the fungus Alternaria solani, however other Alternaria species are known to infect plants, such as A. alternata. Early blight is generally observed on mature plants, or those weakened from dehydration or lack of nutrients, however it may also develop on seedlings. The disease manifests as black or brown lesions on leaves which expand with a yellow hallo and may have dark concentric rings (“bullseye” type spots). As the lesions expand it leads to defoliation due to the death of the affected leaves. As a result, infected plants produce sun-scalded as well as poor-colored fruits. Infected tubers present with dark-colored sunken lesions.

Current treatment and prevention of early blight infection relies on chemical fungicides, such as Mancozeb and chlorothalonil. However, A. solani is becoming increasing resistant to some chemical fungicide treatments (Bauske, M. J., Mallik, I., Yellareddygari, S .K. R., and Gudmestad, N. C. 2018(a). Spatial and temporal distribution of mutations conferring QoI and SDHI resistance in Alternaria solani across the United States. Plant Dis. 102:349-358).

There are currently no commercial potato cultivars completely resistant to early blight. However, resistance has been observed in wild varieties of potato (Solanum commersonii). One such example is S. commersonii accession CGN18024_1.

S. commersonii CGN18024_1 was found to be resistant to four Alternaria solani isolates—T2R3B, ConR4D, ConR2J and ConR1H. These A. solani isolates were collected in the US by Applicant, were sent for genome sequencing. The reads were assembled and comparison with the altNL03003 genome showed that the genomes were well assembled (>95% of complete genome assembled, largest contigs >2Mb). All 31 effector candidates, except AsEC1, were found back in the isolates from the US. Resistance to fungicides through mutations known to occur in Alternaria can also be assessed through these genome sequences.

### Mapping the A. solani Resistant Trait

To fine map the resistance locus within S. commersonii, progeny of four intraspecies crosses (within Solanum commersonii) were tested for resistance to Alternaria solani. A clear segregation of resistance was found in the populations, and bulks of susceptible and resistant progeny for BSA-RNAseq analysis in the best-characterized population AJW12 (CGN1802_1×CGN18024_3) were prepared. The remaining populations were also tested to confirm their phenotypes.

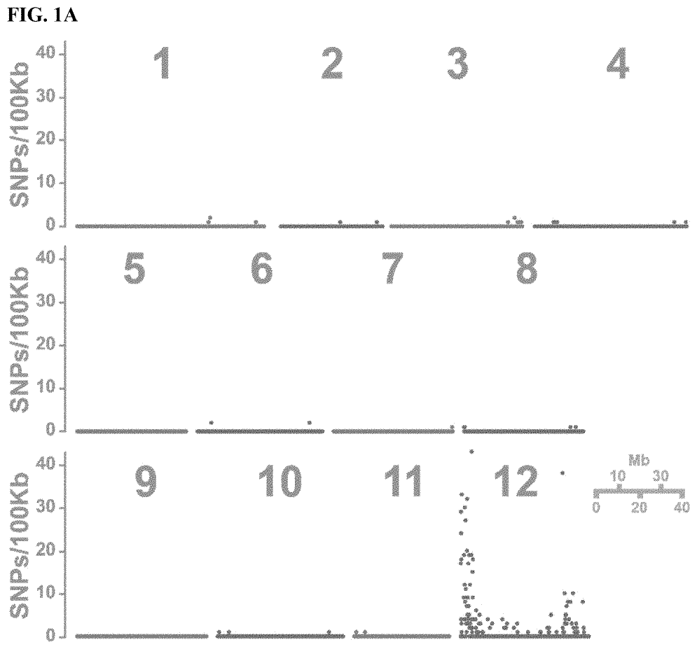

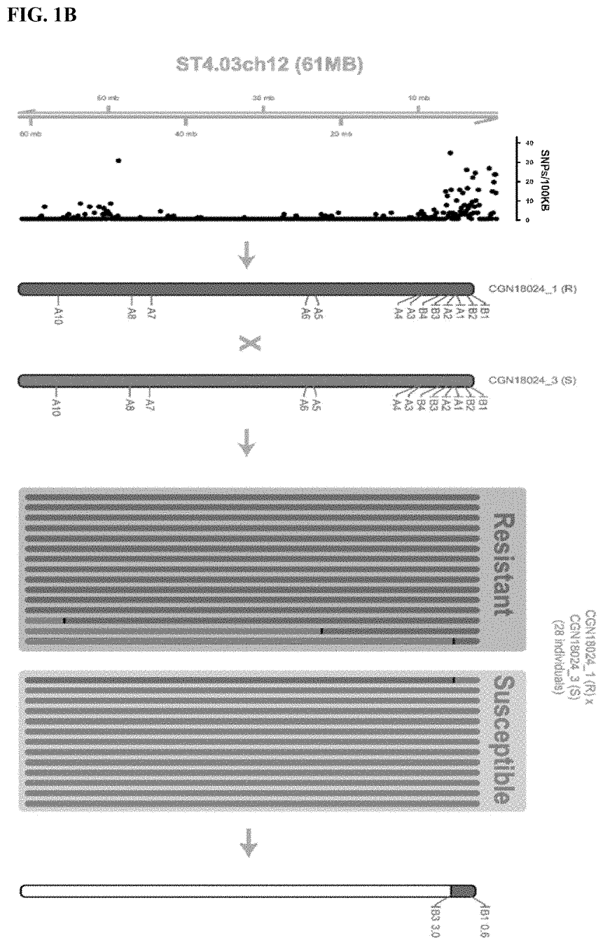

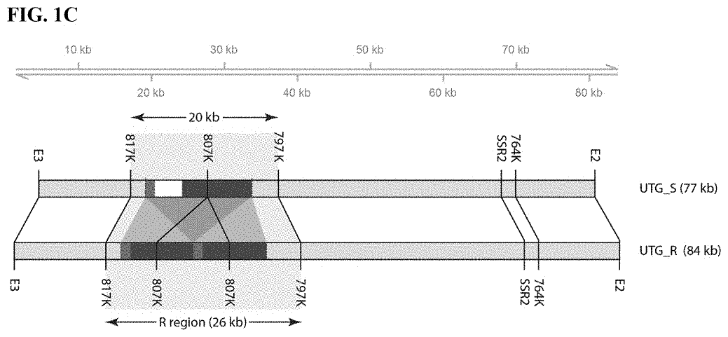

RNA samples of the parents of population AJW12 as well as pools of resistant and susceptible progeny were sent for sequencing and the resulting sequences were analyzed. Putative SNPs linked to resistance were identified by aligning the RNAseq reads to the DMv3.04 genome, after which the SNPs were filtered to identify SNPs that are heterozygous only in the resistant parent and AJW12 progeny (being absent in the susceptible parent and susceptible progeny of AJW12). The filtered SNPs were plotted in windows of 100 Kb over the 12 potato chromosomes (FIG. 1A). This analysis revealed that almost all filtered SNPs map to chromosome 12, indicating that resistance to A. solani is located on that chromosome.

Next, a selection of SNPs broadly covering chromosome 12 were used to develop markers for use in a high-resolution melting (HRM) analysis to genotype the parents of AJW12 and a selection of 28 progeny genotypes (15 resistant and 13 susceptible). Doing so, SNP markers linked to resistance were identified. Moreover, 4 recombinants were found. This limited the genetic window for the resistance to the top of chromosome 12, between SNP markers ‘B1’ and ‘B3’, corresponding to a region of approximately 3 Mb based on the homologous chromosome from the DMv4.03 genome (FIG. 1B). This region contains many genes, including 2 R gene clusters.

The genome of resistant Solanum commersonii accession CGN18024_1 was sequenced using a combination of Oxford Nanopore (ONT) and DNBseq technology. The CGN18024_1 draft assembly was of high quality and a big improvement over the existing S. commersonii (‘Cmmlt’) genome.

The CGN18024_1 resistance gene was mapped to a region of ˜80 kb in the homologous DMv4.03 genome sequence. The corresponding region in CGN18024_1 is about the same size. By aligning the nanopore reads from CGN18024_1 to the draft genome assembly, a genome rearrangement was found in the resistant haplotype of CGN18024_1 that is absent in the susceptible haplotype.

Several rounds of polishing on the CGN18024_1 genome assembly were performed, resulting in a further improvement of the BUSCO completeness score (95.7%, Table 1 below). Next, BUSCO was used to train Augustus gene prediction software to generate a draft gene prediction on the CGN18024_1 genome. Alignment of ONT reads from CGN18024_1 to the CGN18024_1 genome showed evidence for a structural rearrangement in the resistance region. The heterozygous nature of this rearrangement made it possible to separate ONT reads covering the different haplotypes, such that the resistant and susceptible haplotypes were assembled separately. These assemblies were then separately polished with DNB reads and aligned using nucmer. The analysis revealed a duplication of approximately 9.9 kb in the resistant haplotype (UTG_R). The corresponding (non-duplicated) region in the susceptible haplotype (UTG_S) contains an insertion of around 3.7 kb (FIG. 1C). Consequently, the resistance region in the resistant haplotype is about 6.2 kb bigger than the susceptible haplotype.

Shown in Table 1 are properties of genome assemblies of DMv4.03 (doubled monohaploid Solamum tuberosum Group Phureja clone DM), Solyntus (diploid S. tuberosum), Cmmlt (publicly available S. commersonii genome) and 18024_1v5.2 (the polished genome assembly of S. commersonii CGN18024_1, underlined).

TABLE 1
Comparison of statistics of different potato genome assemblies.

| | DMv4.03 | Solyntus | CMM1t | 18024_1v5.2 |
|---|---|---|---|---|
| Year | 2011 | 2019 | 2015 | 2020 |
| Contigs | >3500 | 185 | 278460 | 637 |
| #N's (Mb) | 155 (DM4.04) | 0.015 | 8 | 0 |
| N50 (Mb) | 0.031 | 7.55 | 0.007 | 4 |
| Largest contig | | | 0.17 | 21 |
| (Mb) | | | | |
| BUSCO (%) | 96.1 | 94.6 | 81.9 | 95.7 |

Besides the major rearrangement, several other minor insertion/deletions were found when UTG_R and UTG_S were compared. These differences were used to develop new PCR markers linked to resistance. Combined with a test for early blight resistance, on recombinant genotypes from population AJW12 (CGN18024_1×CGN18024_3), it was possible to further reduce the resistance region. The region is now delimited by markers ‘797K’ and ‘817K’, corresponding to a region of around 26 and 20 kb in the resistant and susceptible haplotype of CGN18024_1 respectively (FIG. 1C).

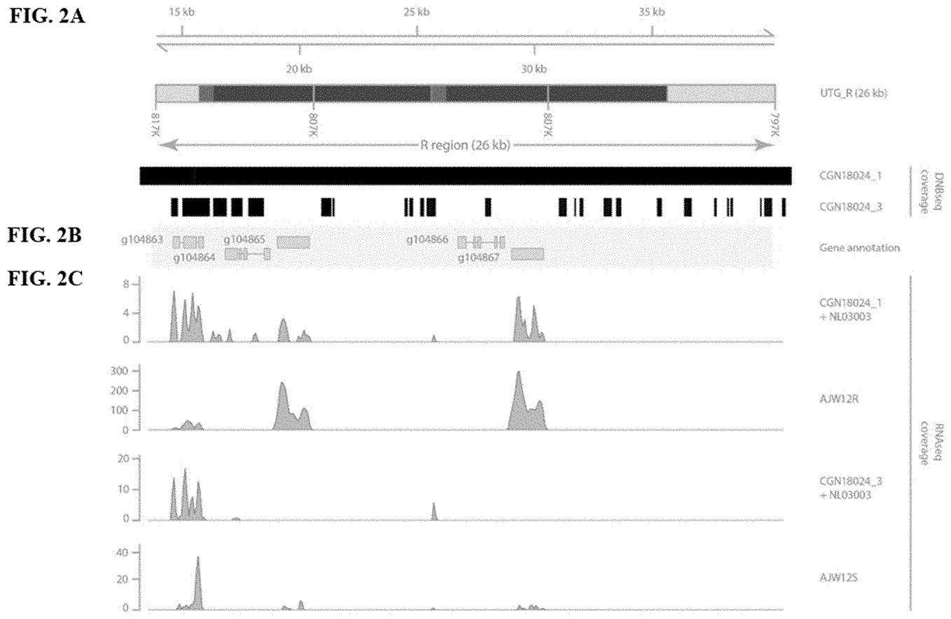

Alignment of DNBseq reads from CGN18024_1 and CGN18024_3 to UTG R confirms that the resistance region is fully covered by reads from CGN18024_1 whereas only few reads from CGN18024_3 map to this contig (FIG. 2A).

Surprisingly, no resistance genes were identified in the resistance region, or even close to the region. Instead, alignment of RNAseq data from CGN18024_1/18024_3 and pools of resistant/susceptible AJW12 progeny to UTG_R points to a different type of candidate gene in the resistance region. A cluster of putative glycosyltransferases (GTs) was found in the resistance region of CGN18024_1. Predicted transcripts g104865 and g104867 (FIG. 2B) are located between markers ‘797K’ and ‘817K’ on UTG R. They are both highly expressed in the resistant pool of AJW12, whereas expression in the susceptible pool is virtually non-detected. Moreover (low) expression of those genes is detected in samples of CGN18024_1, but not in CGN18024_3 samples (FIG. 2C). Candidate genes g104865 and g104867 both code for putative glycosyltransferases (GTs). Because of the rearrangement, one of the predicted GTs got duplicated. These duplicated genes (ScGTR1 and ScGTR2) are the most likely resistance gene candidates in CGN18024_1.

Interestingly, most S. commersonii genotypes that were tested (28/39) contained the duplication, but not all are resistant (11/39 genotypes contain the duplication but are susceptible). Moreover, some resistant genotypes (3/39) do not have the duplication. Thus, while, the duplication is 100% linked to resistance in progeny from CGN18024_1, this is not the case for other S. commersonii accessions. It is possible that alternative mechanisms lead to resistance of these S. commersonii accessions.

In some embodiments, the disclosure relates to a sequence fragment of at least 15 consecutive nucleotides of the ScGTR1 gene described by SEQ ID NO: 3 or its complementary sequence. In some embodiments, the fragment is isolated. In some embodiments, the disclosure relates to a method for identifying a plant comprising a ScGTR1 allele, comprising using said fragment.

In some embodiments, the disclosure relates to a sequence fragment of at least 15 consecutive nucleotides of the ScGTR2 gene described by SEQ ID NO: 4 or its complementary sequence. In some embodiments, the fragment is isolated. In some embodiments, the disclosure relates to a method for identifying a plant comprising a ScGTR2 allele, comprising using said fragment.

### Transient Expression of ScGTR1 and ScGTR2

To investigate if the two glycosyltransferases (ScGTR1 and ScGTR2) from resistant S. commersonii genotype CGN18024_1 are indeed involved in resistance, a transient assay was performed. The genes were cloned in expression vectors for overexpression in plants. Next, they were transiently expressed in leaves of CGN18024_1 (resistant S. commersonii), CGN18024_3 (susceptible S. commersonii), Atlantic and Bintje (both susceptible S. tubersum varieties). The infiltrated spots were inoculated with spores from A. solani two days after agroinfiltration. Sizes of the lesions were measured 5 days post inoculation as a measure for disease resistance. Lesion sizes smaller than 3 mm were considered resistant as they did not exceed beyond inoculation droplet.

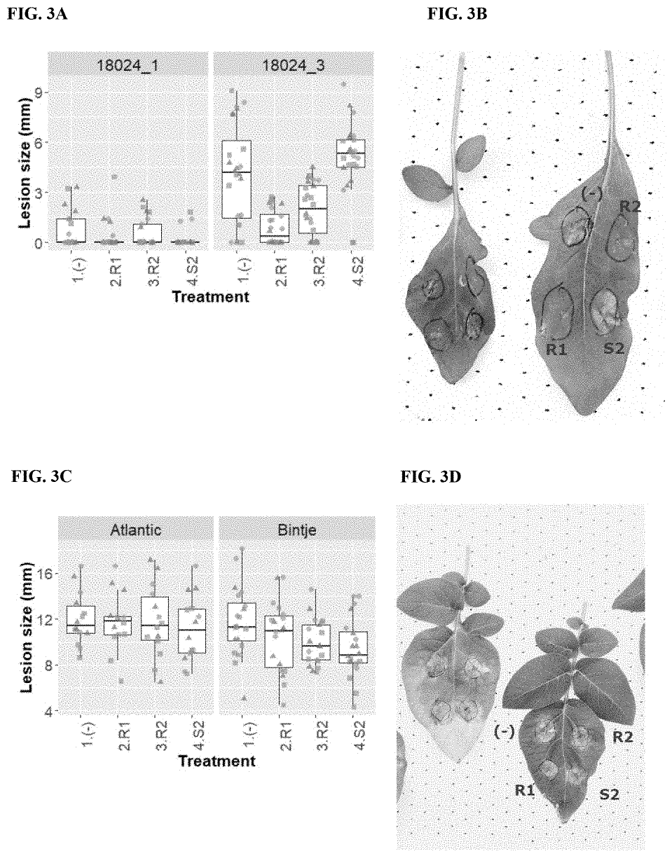

As shown in FIG. 3A, CGN18024_1 remained resistant in all cases, while large lesions developed on leaves of CGN18024_3 where it was infiltrated with an empty vector (‘-’) or the (non-functional) GT allele (ScGTS2) from the susceptible haplotype (‘S2’) (FIG. 3B shows two leaves of the same age). Lesions on the spots where ScGTR1 and ScGTR2 were expressed (‘R1’ and ‘R2’) were much smaller compared to these controls (FIG. 3A & FIG. 3B). This shows that expression of ScGTR1 and ScGTR2 in susceptible S. commersonii can indeed affect resistance to A. solani.

Interestingly, as shown in FIG. 3C & FIG. 3D, there was no clear difference between the different spots that were infiltrated in Atlantic or Bintje. These results suggested that resistance may be through modification (i.e., glycosylation) of a host-specific compound.

### Anti-Fungal Activity Via a Host-Specific Compound

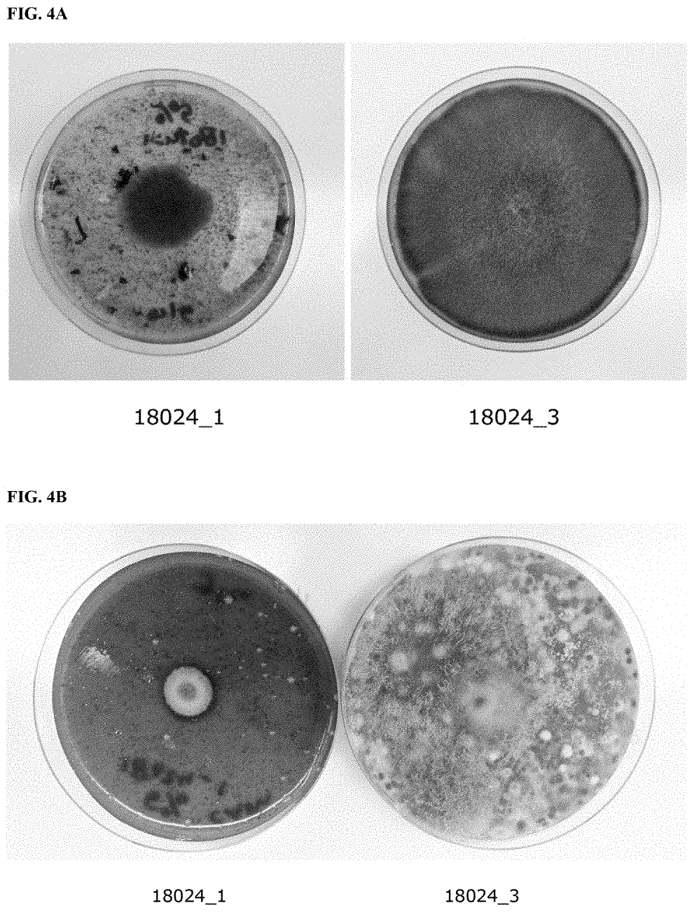

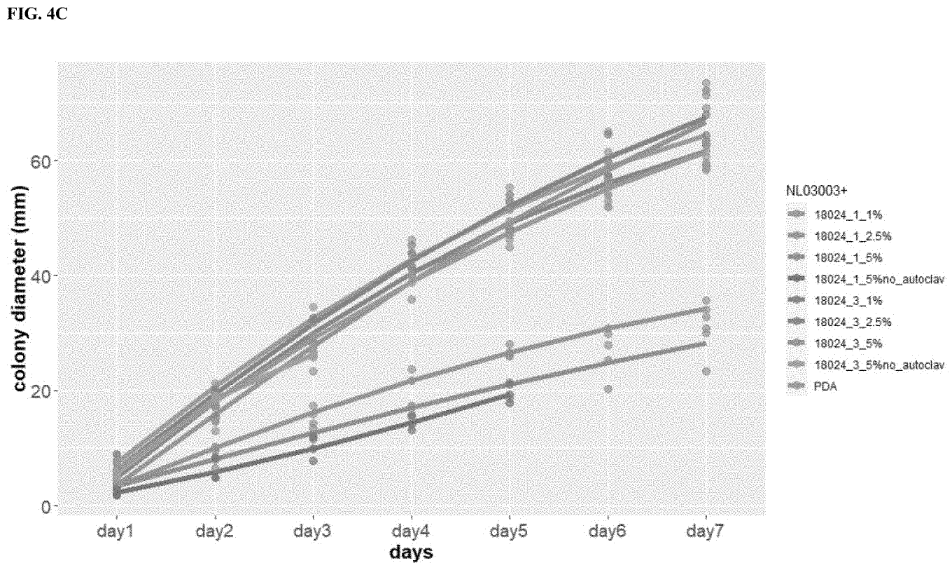

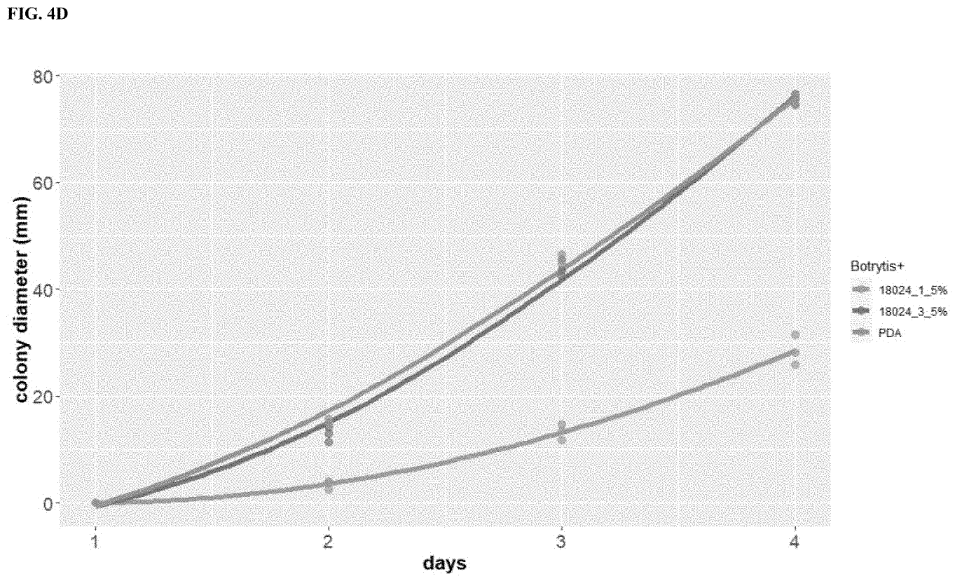

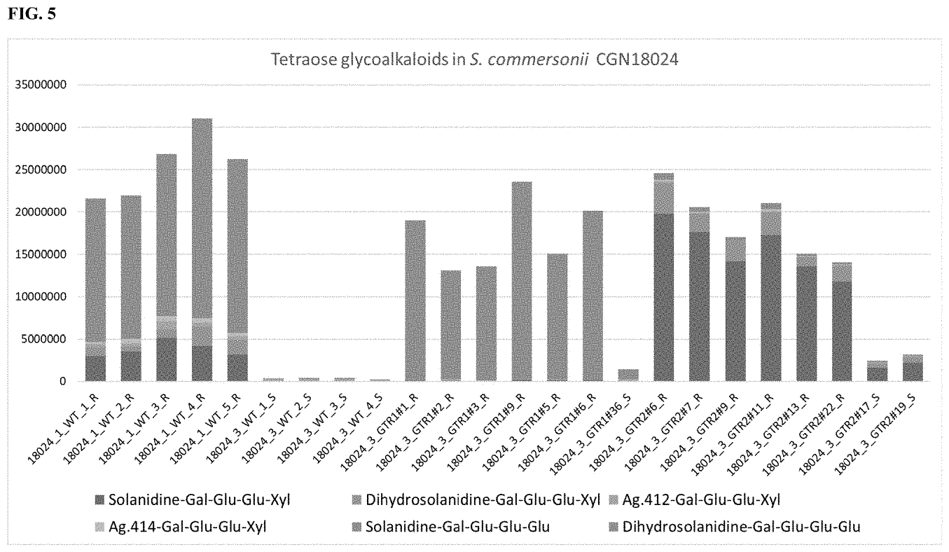

To investigate this, leaf extracts from susceptible and resistant S. commersonii (CGN18024_3 and CGN1802_1) were prepared. The extracts were semi-sterilized (by incubating at 60° C. for 15 min) or autoclaved and added to PDA plates in different concentrations. A PDA plug containing mycelium from A. solani was placed at the center of each plate, and colony growth was monitored in the following days. Strikingly, the CGN18024_1 extract strongly inhibited growth of A. solani at concentrations >2.5% (FIG. 4A, left image). On plates containing extract from CGN18024_3, colony growth was unchanged compared to normal PDA plates (FIG. 4A, right image). The inhibitory effect of the plant extract was not affected by autoclaving (see also (FIG. 4C).

A few days after A. solani was plated, fungal contamination appeared on the plates containing semi-sterilized leaf extract from CGN18024_3. Remarkably, such contamination did not appear on the plates containing extract of CGN18024_1 (FIG. 4B). Thus, the compound from CGN18024_1 does not just inhibit growth of A. solani, but growth of other fungi as well.

The agroinfiltration experiments and the test with plant extracts gave some important insights in the mechanism that leads to A. solani resistance in CGN18024_1; resistance appears to be caused by a host-specific metabolite that is heat-stable. Moreover, this compound has broad anti-fungal activity that is dependent on glycosylation. Previous experiments showed that the glycosyltransferases involved in the resistance are mainly expressed in young tissue and not upregulated upon pathogen infection (data not shown). These observations all point to a specific class of compounds, the glycoalkaloids.

### ScGTR1 and ScGTR2 Transgenic Plants

To better study the effect of ScGTR1 and ScGTR2 on resistance of S. commersonii, and possibly S. tuberosum, susceptible S. commersonii 18024_3 and S. tuberosum Atlantic were transformed with overexpression constructs for both genes. Greater than 40 transformants were obtained for each transformation. A disease screen was performed on the transformants to evaluate resistance to A. solani, and the data confirmed the results that were obtained earlier in the transient assay. ScGTR1 and ScGTR2 did not confer resistance to Atlantic, whereas almost all 18024_3 transformants were found to be resistant. Just one out of 37 18024_3 transformants with ScGTR1 was found to be susceptible and only 2 out of 33 18024_3 ScGTR2 transformants displayed susceptibility.

### Metabolite Analysis of Susceptible and Resistant Plants

Wildtype 18024_1 (Resistant; 18024_1 wt), wildtype 18024_3 (Susceptible; 18024_3 wt) and 7 transformants from each transformation (ScGTR1 and ScGTR2) were selected for a metabolite analysis. All resistant genotypes (18024_1 wt, 18024_3 GTR1, 18024_3 GTR2) contained tetraose glycoalkaloids (composed of an aglycone conjugated to an oligosaccharide containing 4 monosaccharide moieties), whereas susceptible genotypes (18024_3 wt) contain only triose glycoalkaloids (containing 3 monosaccharide moieties instead of 4), or very low levels of tetraose glycoalkaloids. Specifically, two distinct tetraoses were identified on glycoalkaloids of CGN18024_1: Gal-Glu-Glu-Glu and Gal-Glu- Glu-Xyl. Interestingly, the ScGTR1 transformants contain only Gal-Glu-Glu-Glu-alkaloids, whereas ScGTR2 transformants contain almost exclusively Gal-Glu-Glu-Xyl alkaloids (FIG. 5). Thus, it appears that ScGTR1 is responsible for the glucosylation of a triose glycoalkaloid precursor in S. commersonii, while ScGTR2 catalyses xylosilation of the same substrate. Both types of conversions seem to confer resistance to A. solani in S. commersonii. Structures of the triose and tetraose glycoalkaloids are shown below in Table 2.

TABLE 2
triose and tetraose glycoalkaloids
Triose glycoalkaloids from S. tuberosum

| | |
|---|---|
| Solanidine-Gal-Glu-Rha Also known as α-solanine | 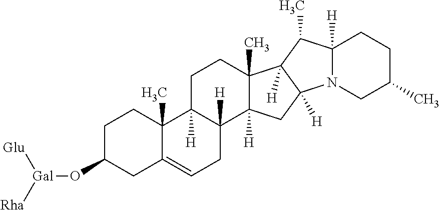 |
| Solanidine-Gal-Rha-Rha Also known as α-chaconine | 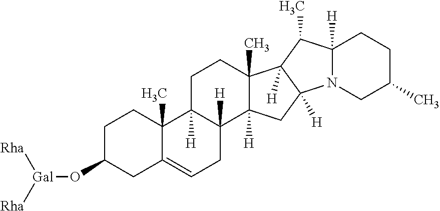 |

Tetraose glycoalkaloids from S. commersonii

| | |
|---|---|
| Solanidine-Gal-Glu-Glu-Glu: (dehydro-) commersonine produced by ScGTRl | 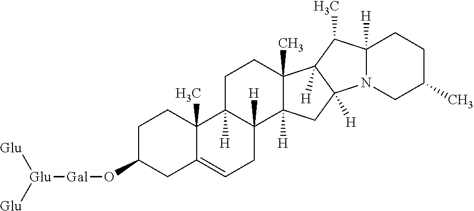 |
| Solanidine-Gal-Glu-Glu-Xyl: (dehydro-) demissine pro- duced by ScGTR2 | 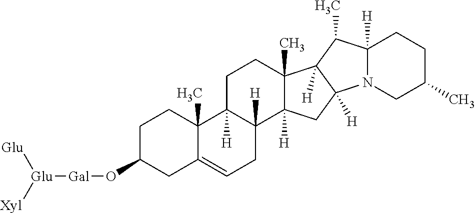 |

Thus, it appears that A. solani resistance in S. commersonii is caused by modification of a glycoalkaloid. The addition of an extra glycose (glucose or xylose) converts it to a version that strongly inhibits A. solani as well as other fungi. The biosynthesis pathway that leads to the production of glycoalkaloids in potato and tomato is rather conserved and well-characterized and glycoalkaloids have been reported to serve as a protective barrier to a broad range of pathogens and pests (Cárdenas, Sonawane et al. 2015). The specific glycoalkaloids that are identified in S. commersonii have been described to also be effective against pest insects such as Colorado potato beetle as well (Sinden, Sanford et al. 1980).

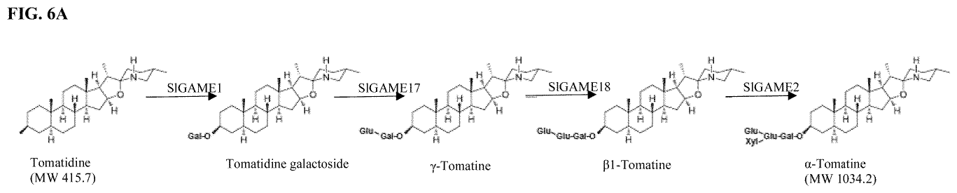

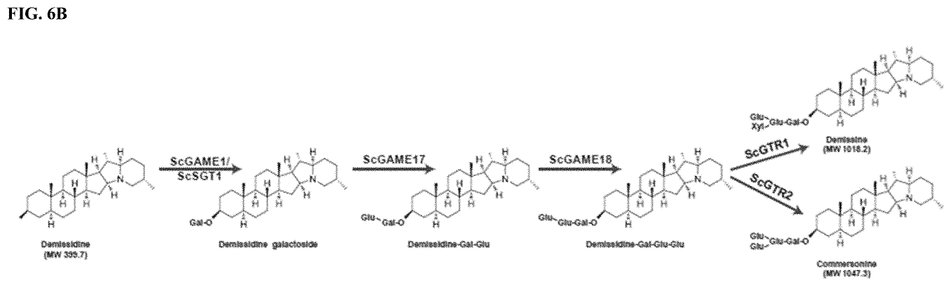

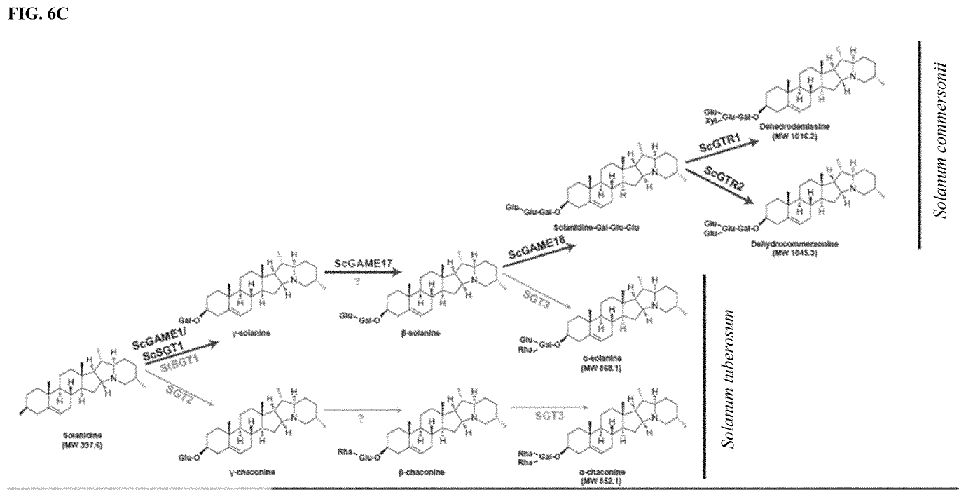

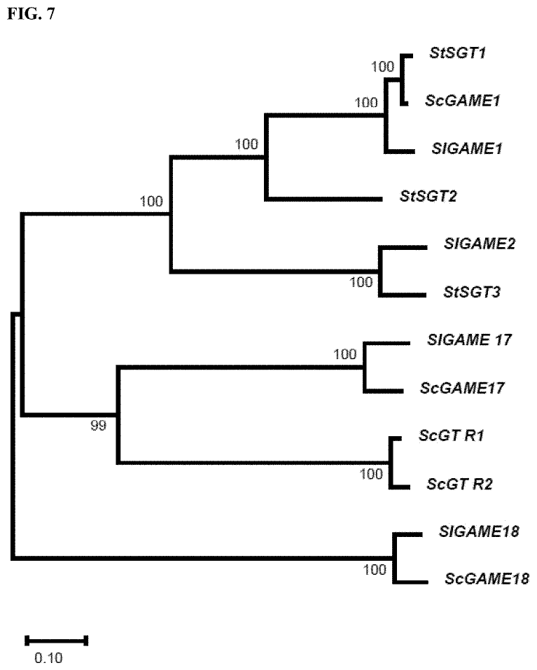

Glycoalkaloids from S. commersonii and S. tuberosum have identical precursors (the solanidine aglycone, FIG. 6C), and the only difference between the tetraose glycoalkaloids from S. commersonii and the triose glycoalkaloids of S. tuberosum is in the sugar groups.

The glycoalkaloid biosynthesis pathway in tomato and potato is well characterized, and the genes involved are syntenic (FIG. 6A-6C). Combined with the availability of the S. commersonii genome, the missing enzyme(s) in S. tuberosum that would enable ScGTR1 and/or ScGTR2 to produce tetraose glycoalkaloids are homologs of the GAME family, specifically, GAME7, and/or GAME18. As the precursor is already produced in S. tuberosum, it is possible to produce the tetraose glycoalkaloids (and therein impart disease resistance) by making a few modifications in S. tuberosum and other Solanum species that are susceptible to infection.

As shown in FIG. 6C and in Table 2, triose glycoalkaloids α-chaconine and α-solanine from S. tuberosum contain a rhamnose (Rha) and this is not present on the triose glycoalkaloids in S. commersonii. The addition of this rhamnose is the last step in the triose formation for both branches of the potato steroidal glycoalkaloid biosynthetic pathway in S. tuberosum. The gene that produces the enzyme response for this, β-steroidal glycoalkaloid rhamnosyltransferase, is known as Sgt3 (GenBank accession no. DQ266437). Potato Sgt3 antisense transgenic lines wherein production of α-chaconine and α-solanine is reduced have been produced and are known in the art (McCue, K. F., et al. 2007. Potato glycosterol rhamnosyltransferase, the terminal step in triose side chain biosynthesis. Phytochemistry 68:327-334). Thus, in addition to expression of ScGTR1 and/or ScGTR2 with functional orthologs of GAME17 and/or GAME18, S. tuberosum may be further modified to reduce expression of Sgt3 and/or block activity of (3-steroidal glycoalkaloid rhamnosyltransferase.

This way, Solanum species could be engineered to produce the tetraose glycoalkaloids in leaves specifically, which has the potential to provide broad-spectrum resistance against a range of foliar microbial pathogens as well as pest insects.

In some embodiments, the disclosure teaches a method for conferring resistance to a pest or pathogen in a Solanum spp. plant, plant part, or plant cell, comprising at least one of: (a) introducing a nucleic acid into a Solanum spp. plant, plant part, or plant cell, wherein said nucleic acid encodes an amino acid sequence at least 90% identical to SEQ ID NO: 1, and (b) introducing a nucleic acid into a Solanum spp. plant, plant part, or plant cell, wherein said nucleic acid encodes an amino acid sequence at least 90% identical to SEQ ID NO: 2. In some embodiments, the nucleic acid of (a) comprises SEQ ID NO: 3, or a sequence at least 75% identical thereto. In some embodiments, the nucleic acid of (b) comprises SEQ ID NO: 4, or a sequence at least 75% identical thereto. In some embodiments, the Solanum spp. plant, plant part, or plant cell, comprises an Sgt3 gene knockout and/or reduced Sgt3 expression.

In some embodiments, the method further comprises introducing a nucleic acid encoding a functional homolog of a glycoalkaloid metabolism (GAME) polypeptide. In some embodiments, the functional homolog of a glycoalkaloid metabolism (GAME) polypeptide is a glycosyltransferase. In some embodiments, the glycosyltransferase is a functional homolog of GAME18 and/or GAME17. In some embodiments, the glycosyltransferase is ScGAME18. In some embodiments, the glycosyltransferase is a functional homolog of GAME17. In some embodiments, the glycosyltransferase is ScGAME17. In some embodiments, the nucleic acid encodes an amino acid sequence at least 90% identical to SEQ ID NO: 10. In some embodiments, the nucleic acid comprises SEQ ID NO: 9, or a sequence at least 75% identical thereto. In some embodiments, the nucleic acid encodes an amino acid sequence at least 90% identical to SEQ ID NO: 12. In some embodiments, the nucleic acid comprises SEQ ID NO: 11, or a sequence at least 75% identical thereto.

In some embodiments, the method further comprises introducing an antisense Sgt3 transgene. In some embodiments, the method comprises knocking out the Sgt3 gene.

In some embodiments, the expression of at least one of the nucleic acids generates tetraose glycoalkaloids, and the tetraose glycoalkaloids confers resistance to a pest or pathogen. In some embodiments, the Solanum spp. plant is resistant to at least one of the following fungal pathogens: Alternaria spp., Colletotrichum coccodes, Rhizoctonia solani Fusarium spp., Phoma spp., Helminthosporium solani, Polyscytalum pustulans, Verticillium spp., Helicobasidium mompa, and Synchytrium endobioticum. In some embodiments, the fungal pathogen is Alternaria alternata or Alternaria solani. In some embodiments, the Solanum spp. plant is S. tuberosum, S. melongena, or S. lycopersicum.

In some embodiments, the disclosure relates to a sequence fragment of at least 15 consecutive nucleotides of the ScGAME18 gene described by SEQ ID NO: 9 or its complementary sequence. In some embodiments, the fragment is isolated. In some embodiments, the disclosure relates to a method for identifying a plant comprising a ScGAME18 allele, comprising using said fragment.

In some embodiments, the disclosure relates to a sequence fragment of at least 15 consecutive nucleotides of the ScGAME17 gene described by SEQ ID NO: 11 or its complementary sequence. In some embodiments, the fragment is isolated. In some embodiments, the disclosure relates to a method for identifying a plant comprising a ScGAME17 allele, comprising using said fragment.

In some embodiments, the disclosure relates to a Solanum spp. plant produced by the methods disclosed herein.

### Methods for Generating Resistance to Pest And Pathogens and Plants Produced Therefrom

### Expression Vectors for Potato Transformation

In some embodiments, the disclosure relates to a plant transformation vector designated pSIMScGTR1 comprising the nucleotide sequence set forth in SEQ ID NO: 3, and wherein the plant transformation vector was deposited under ATCC Accession No. PTA-______.

In some embodiments, the disclosure relates to a plant transformation vector designated pSIMScGTR2 comprising the nucleotide sequence set forth in SEQ ID NO: 4, and wherein the plant transformation vector was deposited under ATCC Accession No. PTA-______.

In some embodiments, the disclosure relates to a plant transformation vector designated pSIML2GGA1 comprising the nucleotide sequences set forth in SEQ ID NOs: 3 and 9, and wherein the plant transformation vector was deposited under ATCC Accession No. PTA-______.

A plant transformation vector designated pSIML2GGA2 comprising the nucleotide sequences set forth in SEQ ID NOs: 4 and 9, and wherein the plant transformation vector was deposited under ATCC Accession No. PTA-______.

A plant transformation vector designated pSIML2GGA3 comprising the nucleotide sequences set forth in SEQ ID NOs: 3, 4, and 9, and wherein the plant transformation vector was deposited under ATCC Accession No. PTA-______.

In some embodiments, the disclosure relates to a plant transformation vector comprising at least one of: a nucleotide sequence encoding an amino acid sequence at least 90% identical to SEQ ID NO: 1, a nucleotide sequence encoding an amino acid sequence at least 90% identical to SEQ ID NO: 2; a nucleotide sequence encoding an amino acid sequence at least 90% identical to SEQ ID NO: 10; and a nucleotide sequence encoding an amino acid sequence at least 90% identical to SEQ ID NO: 12. In some embodiments, the plant transformation vector further comprises an antisense Sgt3 transgene.

In some embodiments, the present disclosure provides a nucleic acid sequence that encodes an amino acid sequence that shares at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 1, and transgenic plants comprising said sequences.

In some embodiments, the present disclosure provides a nucleic acid sequence that encodes an amino acid sequence that shares at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 2, and transgenic plants comprising said sequences.

In some embodiments, the present disclosure provides a nucleic acid sequence that shares at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 3, and transgenic plants comprising said sequences.

In some embodiments, the present disclosure provides a nucleic acid sequence that shares at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 4, and transgenic plants comprising said sequences.

In some embodiments, the present disclosure provides a nucleic acid sequence that encodes an amino acid sequence that shares at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 10, and transgenic plants comprising said sequences.

In some embodiments, the present disclosure provides a nucleic acid sequence that encodes an amino acid sequence that shares at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 12, and transgenic plants comprising said sequences.

In some embodiments, the present disclosure provides a nucleic acid sequence that shares at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 9, and transgenic plants comprising said sequences.

In some embodiments, the present disclosure provides a nucleic acid sequence that shares at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, or 100% sequence identity to SEQ ID NO: 11, and transgenic plants comprising said sequences.

Methods of alignment of sequences for comparison are well known in the art. Various programs and alignment algorithms are described in: Smith and Waterman (Adv. Appl. Math., 2:482, 1981); Needleman and Wunsch (J. Mol. Biol., 48:443, 1970); Pearson and Lipman (Proc. Natl. Acad. Sci., 85:2444, 1988); Higgins and Sharp (Gene, 73:237 44, 1988); Higgins and Sharp (CABIOS, 5:151 53, 1989); Corpet et al. (Nuc. Acids Res., 16:10881 90, 1988); Huang et al. (Comp. Appls Biosci., 8:155 65, 1992); and Pearson et al. (Meth. Mol. Biol., 24:307 31, 1994). Altschul et al. (Nature Genet., 6:119 29, 1994) presents a detailed consideration of sequence alignment methods and homology calculations.

In some embodiments, the disclosure relates to plants and plant parts transformed with the vectors disclosed herein. In some embodiments, the plant is resistant to a fungal pathogen.

Additional examples of potato transformation vectors are well known in the art. See for example, U.S. Pat. Nos. 9,328,352, 9,873,885, 8,710,311, 8,754,303, 8,889,963, 9,918,441, 8,889,964, 9,909,141, 9,924,647, and 9,968,043, which are all incorporated herein by reference.

### Expression Vectors for Potato Transformation: Marker Genes

Expression vectors usually include at least one genetic marker, operably linked to a regulatory element (a promoter, for example) that allows transformed cells containing the marker to be either recovered by negative selection, i.e., inhibiting growth of cells that do not contain the selectable marker gene, or by positive selection, i.e., screening for the product encoded by the genetic marker. Many commonly used selectable marker genes for plant transformation are well known in the transformation arts, and include, for example, genes that code for enzymes that metabolically detoxify a selective chemical agent which may be an antibiotic or an herbicide, or genes that encode an altered target which is insensitive to the inhibitor. A few positive selection methods are also known in the art.

One commonly used selectable marker gene for plant transformation is the neomycin phosphotransferase II (nptII) gene which, when under the control of plant regulatory signals, confers resistance to kanamycin. Fraley et al., Proc. Natl. Acad. Sci. U.S.A., 80:4803 (1983). The aminoglycoside phosphotransferases APH(3′)II and APH(3′)I carried by transposons TnS and Tn601 respectively were shown to inactivate the related aminoglycoside antibiotics G418), neomycin and kanamycin (Davies and Smith, 1978; Jimenez and Davies, 1980). The kanamycin resistance (KmR) gene from Staphylococcus aureus, which are used for the present disclosure, was sequenced and identified when compared with similar genes isolated from Streptomyces fradiae and from two transposons, Tn5 and Tn903, originally isolated from Klebsiella pneumoniae and Salmonella typhimurium, respectively (Gray and Fitch, 1983). Another commonly used selectable marker gene is the hygromycin phosphotransferase gene which confers resistance to the antibiotic hygromycin. Vanden Elzen et al., Plant Mol. Biol., 5:299 (1985).

Additional selectable marker genes of bacterial origin that confer resistance to antibiotics include gentamycin acetyl transferase, streptomycin phosphotransferase and aminoglycoside-3′-adenyl transferase, the bleomycin resistance determinant. Hayford et al., Plant Physiol. 86:1216 (1988), Jones et al., Mol. Gen. Genet., 210:86 (1987), Svab et al., Plant Mol. Biol. 14:197 (1990) Hille et al., Plant Mol. Biol. 7:171 (1986). Other selectable marker genes confer resistance to herbicides such as glyphosate, glufosinate or bromoxynil. Comai et al., Nature 317:741-744 (1985), Gordon-Kamm et al., Plant Cell 2:603-618 (1990) and Stalker et al., Science 242:419-423 (1988).

Selectable marker genes for plant transformation not of bacterial origin include, for example, mouse dihydrofolate reductase, plant 5-enolpyruvylshikimate-3-phosphate synthase and plant acetolactate synthase. Eichholtz et al., Somatic Cell Mol. Genet. 13:67 (1987), Shah et al., Science 233:478 (1986), Charest et al., Plant Cell Rep. 8:643 (1990).

Another class of marker genes for plant transformation requires screening of presumptively transformed plant cells rather than direct genetic selection of transformed cells for resistance to a toxic substance such as an antibiotic. These genes are particularly useful to quantify or visualize the spatial pattern of expression of a gene in specific tissues and are frequently referred to as reporter genes because they can be fused to a gene or gene regulatory sequence for the investigation of gene expression. Commonly used genes for screening presumptively transformed cells include beta-glucuronidase (GUS), beta-galactosidase, luciferase and chloramphenicol acetyltransferase. Jefferson, R. A., Plant Mol. Biol. Rep. 5:387 (1987), Teeri et al., EMBO J. 8:343 (1989), Koncz et al., Proc. Natl. Acad. Sci. USA 84:131 (1987), DeBlock et al., EMBO J. 3:1681 (1984). In vivo methods for visualizing GUS activity that do not require destruction of plant tissue are available. Molecular Probes publication 2908, IMAGENE GREEN, p. 1-4 (1993) and Naleway et al., J. Cell Biol. 115:151a (1991). However, these in vivo methods for visualizing GUS activity have not proven useful for recovery of transformed cells because of low sensitivity, high fluorescent backgrounds and limitations associated with the use of luciferase genes as selectable markers.

In some aspects, a gene encoding Green Fluorescent Protein (GFP) has been utilized as a marker for gene expression in prokaryotic and eukaryotic cells. Chalfie et al., Science 263:802 (1994). GFP and mutants of GFP may be used as screenable markers.

### Expression Vectors for Transformation: Promoters

Genes included in expression vectors are typically driven by a nucleotide sequence comprising a regulatory element, for example, a promoter. Several types of promoters are well known in the transformation arts as are other regulatory elements that can be used alone or in combination with promoters.

As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue-preferred”. Promoters that initiate transcription only in a certain tissue are referred to as “tissue-specific”. A “cell-type” specific promoter primarily drives expression in certain cell types in one or more organs, for example, vascular cells in roots or leaves. An “inducible” promoter is a promoter which is under environmental control. Examples of environmental conditions that may affect transcription by inducible promoters include anaerobic conditions or the presence of light. Tissue-specific, tissue-preferred, cell type specific, and inducible promoters constitute the class of “non-constitutive” promoters. A “constitutive” promoter is a promoter that is active under most environmental conditions, and cell types.

### A. Inducible Promoters

An inducible promoter is operably linked to a gene for expression in a plant. Optionally, the inducible promoter is operably linked to a nucleotide sequence encoding a signal sequence which is operably linked to a gene for expression in a plant. With an inducible promoter the rate of transcription increases in response to an inducing agent.

Any inducible promoter can be used in the instant disclosure. See Ward et al., Plant Mol. Biol. 22:361-366 (1993). Exemplary inducible promoters include, but are not limited to, that from the ACEI system which responds to copper (Mett et al., PNAS 90:4567-4571 (1993)); In2 gene from maize which responds to benzenesulfonamide herbicide safeners (Hershey et al., Mol. Gen Genetics 227:229-237 (1991) and Gatz et al., Mol. Gen. Genetics 243:32-38 (1994)) or Tet repressor from Tn10 (Gatz et al., Mol. Gen. Genetics 227:229-237 (1991)). A particularly preferred inducible promoter is a promoter that responds to an inducing agent to which plants do not normally respond. An exemplary inducible promoter is the inducible promoter from a steroid hormone gene, the transcriptional activity of which is induced by a glucocorticosteroid hormone. Schena et al., Proc. Natl. Acad. Sci. USA 88:0421 (1991).

### B. Constitutive Promoters

A constitutive promoter is operably linked to a gene for expression in a plant or the constitutive promoter is operably linked to a nucleotide sequence encoding a signal sequence which is operably linked to a gene for expression in a plant.

Many different constitutive promoters can be utilized in the instant disclosure. Exemplary constitutive promoters include, but are not limited to, the promoters from plant viruses such as the 35S promoter from CaMV (Odell et al., Nature 313:810-812 (1985)) and the promoters from such genes as rice actin (McElroy et al., Plant Cell 2: 163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)).

The ALS promoter, Xbal/Ncol fragment 5′ to the Brassica napus ALS3 structural gene (or a nucleotide sequence similarity to said Xbal/Ncol fragment), represents a particularly useful constitutive promoter. See PCT application WO 96/30530.

In some embodiments, the present disclosure teaches use of a potato ubiquitin promoter (pUbi7) for constitutive expression of a gene of interest. Isolation of a polyubiquitin promoter and its expression in transgenic potato plants is described in Garbarino et al., Plant Physiology 109(4):1371-1378 (1995), which is hereby incorporated by reference in its entirety.

### C. Tissue-Specific or Tissue-Preferred Promoters

A tissue-specific promoter is operably linked to a gene for expression in a plant. Optionally, the tissue-specific promoter is operably linked to a nucleotide sequence encoding a signal sequence which is operably linked to a gene for expression in a plant. Plants transformed with a gene of interest operably linked to a tissue-specific promoter produce the protein product of the transgene exclusively, or preferentially, in a specific tissue.

Any tissue-specific or tissue-preferred promoter can be utilized in the instant disclosure. Exemplary tissue-specific or tissue-preferred promoters include, but are not limited to, a root-preferred promoter—such as that from the phaseolin gene (Murai et al., Science 23:476-482 (1983) and Sengupta-Gopalan et al., Proc. Natl. Acad. Sci. USA 82:3320-3324 (1985)); a leaf-specific and light-induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)) or a microspore-preferred promoter such as that from apg (Twell et al., Sex. Plant Reprod. 6:217-224 (1993)).

### Expression Vectors for Potato Transformation: Terminators

As used herein, the term “terminator” or “termination sequence” generally refers to a 3′ flanking region of a gene that contains nucleotide sequences which regulate transcription termination and typically confer RNA stability.

The disclosure provides terminator sequences that find use in proper transcriptional processing of recombinant nucleic acids in the transformation vectors taught herein. Although terminator sequences do not by themselves initiate gene transcription, their presence can increase accurate processing and termination of the RNA transcript, and result in message stability. The use of recombinant terminator sequences is established in the art. It is appreciated that an understanding of the molecular mechanisms underlying terminator sequence activity are not required to make or use the present disclosure.

### Methods for Potato Transformation

Numerous methods for plant transformation have been developed and are well known in the art, including biological and physical plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc. Boca Raton, 1993) pages 67-88. In addition, expression vectors and in-vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pages 89-119.

### A. Agrobacterium-Mediated Transformation

One method for introducing an expression vector into plants is based on the natural transformation system of Agrobacterium. See, for example, Horsch et al., Science 227:1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant Sci. 10:1 (1991). Descriptions of Agrobacterium vector systems and methods for Agrobacterium-mediated gene transfer are provided by Gruber et al., supra, Miki et al., supra and Moloney et al., Plant Cell Reports 8:238 (1989). See also, U.S. Pat. No. 5,563,055 (Townsend and Thomas), issued Oct. 8, 1996. There are numerous patents governing Agrobacterium mediated transformation and particular DNA delivery plasmids designed specifically for use with Agrobacterium—for example, U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. No. 5,731,179, EP068730, WO9516031, U.S. Pat. No. 5,693,512, U.S. Pat. No. 6,051,757 and EP904362A1, which are all hereby incorporated by reference in their entirety.

Agrobacterium-mediated plant transformation involves as a first step the placement of DNA fragments cloned on plasmids into living Agrobacterium cells, which are then subsequently used for transformation into individual plant cells. Agrobacterium-mediated plant transformation is thus an indirect plant transformation method. Alternatively, Agrobacterium-mediated plant transformation involves cloning DNA fragments into the disarmed Ti or Vi plasmid of Agrobacterium, such as described in Collier (2018), and using the resulting engineered Agrobacterium for plant transformation.

Agrobacterium-mediated transformation is achieved through the use of a genetically engineered soil bacterium belonging to the genus Agrobacterium. Several Agrobacterium species mediate the transfer of a specific DNA known as “T-DNA” that can be genetically engineered to carry any desired piece of DNA into many plant species. The major events marking the process of T-DNA mediated pathogenesis are: induction of virulence genes, processing and transfer of T-DNA. This process is the subject of many reviews (Ream, 1989; Howard and Citovsky, 1990; Kado, 1991; Hooykaas and Schilperoort, 1992; Winnans, 1992; Zambryski, 1992; Gelvin, 1993; Binns and Howitz, 1994; Hooykaas and Beijersbergen 1994; Lessl and Lanka, 1994; Zupan and Zambryski, 1995).

Agrobacterium-mediated genetic transformation of plants involves several steps. The first step, in which the Agrobacterium and plant cells are first brought into contact with each other, is generally called “inoculation”. Following the inoculation step, the Agrobacterium and plant cells/tissues are usually grown together for a period of several hours to several days or more under conditions suitable for growth and T-DNA transfer. This step is termed “co-culture”. Following co-culture and T-DNA delivery, the plant cells are often treated with bacteriocidal and-or bacteriostatic agents to kill the Agrobacterium.

Although transgenic plants produced through Agrobacterium-mediated transformation generally contain a simple integration pattern as compared to microparticle-mediated genetic transformation, a wide variation in copy number and insertion patterns exists (Jones et al, 1987; Jorgensen et al., 1987). Moreover, even within a single plant genotype, different patterns of transfer DNA integration are possible based on the type of explant and transformation system used (Grevelding et al., 1993). Factors that regulate transfer DNA copy number are poorly understood.

### B. Direct Gene Transfer

Direct plant transformation methods using DNA have also been reported. The first of these to be reported historically is electroporation, which utilizes an electrical current applied to a solution containing plant cells (M. E. Fromm et al., Nature, 319, 791 (1986); H. Jones et al., Plant Mol. Biol., 13, 501 (1989) and H. Yang et al., Plant Cell Reports, 7, 421 (1988).

Another direct method, called “biolistic bombardment”, uses ultrafine particles, usually tungsten or gold, that are coated with DNA and then sprayed onto the surface of a plant tissue with sufficient force to cause the particles to penetrate plant cells, including the thick cell wall, membrane and nuclear envelope, but without killing at least some of them (U.S. Pat. Nos. 5,204,253, US 5,015,580).

A third direct method uses fibrous forms of metal or ceramic consisting of sharp, porous or hollow needle-like projections that literally impale the cells, and also the nuclear envelope of cells. Both silicon carbide and aluminum borate whiskers have been used for plant transformation (Mizuno et al., 2004; Petolino et al., 2000; US5302523 US Application 20040197909) and also for bacterial and animal transformation (Kaepler et al., 1992; Raloff, 1990; Wang, 1995). There are other methods reported, and undoubtedly, additional methods will be developed. The methods taught herein are capable of detecting the non-naturally occurring nucleotide junctions that result from any plant transformation method.

The sequences of the present disclosure may be transferred to any cell, for example, such as a plant cell transformation competent bacterium. Such bacteria are known in the art and may, for instance, belong to the following species: Agrobacterium spp., Rhizobium spp., Sinorhizobium spp., Mesorhizobium spp., Phyllobacterium spp. Ochrobactrum spp. and Bradyrhizobium spp. In some embodiments, such bacteria may belong to Agrobacterium spp.

The present disclosure also relates to a plant cell transforming bacterium comprising the sequences disclosed herein, and which may be used for transforming a plant cell. In some embodiments, the plant transforming bacteria is selected from Agrobacterium spp., Rhizobium spp., Sinorhizobium spp., Mesorhizobium spp., Phyllobacterium spp. Ochrobactrum spp. or Bradyrhizobium spp.

The present disclosure also relates to a method for transforming a plant cell comprising: contacting at least a first plant cell with a plant cell transforming bacteria of the present disclosure; and selecting at least a plant cell transformed with one or more of the sequences disclosed herein. In some embodiments, the plant cell is a potato plant cell. In one embodiment, a method of the disclosure further comprises regenerating a plant from the plant cell.

In some embodiments, the present disclosure provides a method for transforming a plant cell, wherein the method comprises: (i) introducing a plant transformation vector taught herein into the plant cell; and (ii) cultivating the transformed plant cell under conditions conducive to regeneration and mature plant growth. In some embodiments, the plant cell is a potato cell.

The foregoing methods for transformation would typically be used for producing a transgenic variety. The transgenic variety could then be crossed with another (non-transformed or transformed) variety in order to produce a new transgenic variety. Alternatively, a genetic trait that has been engineered into a particular plant line using the foregoing transformation techniques could be moved into another line using traditional backcrossing techniques that are well known in the plant breeding arts. For example, a backcrossing approach could be used to move an engineered trait from a public, non-elite variety into an elite variety, or from a variety containing a foreign gene in its genome into a variety or varieties that do not contain that gene. As used herein, “crossing” can refer to a simple X by Y cross or the process of backcrossing depending on the context.

Persons of ordinary skill in the art will recognize that plants comprising the sequences disclosed herein also includes derivative varieties that retain the essential distinguishing characteristics of the event in question, such as a locus converted plant of that variety or a transgenic derivative having one or more value-added genes incorporated therein (such as herbicide or pest resistance).

Likewise, transgenes can be introduced into the plant using any of a variety of established recombinant methods well-known to persons skilled in the art, such as: Gressel, 1985, Biotechnologically Conferring Herbicide Resistance in Crops: The Present Realities, In Molecular Form and Function of the Plant Genome, L. van Vloten-Doting, (ed.), Plenum Press, New York; Huttner, S. L., et al., 1992, Revising Oversight of Genetically Modified Plants, Bio/Technology; Klee, H., et al., 1989, Plant Gene Vectors and Genetic Transformation: Plant Transformation Systems Based on the use of Agrobacterium tumefaciens, Cell Culture and Somatic Cell Genetics of Plants; Koncz, C., et al., 1986, The Promoter of T. sub.L-DNA Gene 5 Controls the Tissue-Specific Expression of Chimeric Genes Carried by a Novel Type of Agrobacterium Binary Vector; Molecular and General Genetics; Lawson, C., et al., 1990, Engineering Resistance to Mixed Virus Infection in a Commercial Potato Cultivar: Resistance to Potato Virus X and Potato Virus Y in Transgenic Russet Burbank, Bio/Technology; Mitsky, T. A., et al., 1996, Plants Resistant to Infection by PLRV. U.S. Pat. No. 5,510,253; Newell, C. A., et al., 1991, Agrobacterium-Mediated Transformation of Solanum tuberosum L. Cv. Russet Burbank, Plant Cell Reports; Perlak, F. J., et al., 1993, Genetically Improved Potatoes: Protection from Damage by Colorado Potato Beetles, Plant Molecular Biology; all of which are incorporated herein by reference for this purpose.

Methods of modifying nucleic acid constructs to increase expression levels in plants are also generally known in the art (see, e.g. Rogers et al., 260 J. Biol. Chem. 3731-38, 1985; Cornejo et al., 23 Plant Mol. Biol. 567: 81,1993). In engineering a plant system to affect the rate of transcription of a protein, various factors known in the art, including regulatory sequences such as positively or negatively acting sequences, enhancers and silencers, as well as chromatin structure may have an impact. The present disclosure provides that at least one of these factors may be utilized in engineering plants to express a protein of interest. The sequences of the present disclosure are native genetic elements, i.e., are isolated from the selected plant species to be modified.

### Resistance to Other Pests and Pathogens

As will be understood by one skilled in the art, additional expression cassettes may be stacked in the DNA constructs disclosed herein to confer tolerance or resistance to other pests and pathogens, including, but not limited to, late blight, Potato virus Y, Potato virus X, Potato leaf roll virus, tobacco rattle virus, verticillium wilts, and pests such as plant-parasitic nematodes.

Protection from virus such as Potato Virus X and Potato leaf roll virus is achievable employing RNA interference to viral coat protein (CP) sequences. Potato plants engineered to express RNAi constructs to block the production of PVY CP are protected from infection and those expressing RNAi constructs to block the production of Potato leaf roll virus (PLRV) CP were resistant to PLRV (Kaniewski W. K. et al., AgBioForum, 7(1&2): 41-46 (2004)). The present disclosure teaches that RNAi constructs to several viruses could be stacked for robust virus resistance in plants.

Verticillium wilts are vascular wilt diseases caused by soil-borne fungal pathogens that belong to the Verticillium genus. Verticillium dahliae is the most notorious species and can infect hundreds of dicotyledonous hosts including potato. The Vel locus that confers race-specific resistance against Verticillium has been characterized and shown to be effective to resist race 1 isolates of V. dahliae (see U.S. Pat. No. 6,608,245 and Song Y et al., Molecular Plant Pathology 18(2): 195-2009 (2017)). The present disclosure teaches expression of the StVe1 gene for resistance to Verticillium dahlia in plants.

Bacillus thuringiensis (Bt) crystal proteins are pore-forming toxins used as insecticides around the world. Effective use of Bt (or Cry) proteins for Colorado potato beetle resistance was successfully deployed in potato (Kaniewski W. K. et al., AgBioForum, 7(1&2): 41-46 (2004)). The Cry proteins may target the invertebrate phylum Nematoda in several crops (Wei J.Z. et al, PNAS 100(5): 2760-2765 (2003); and Li X.Q. et al. Biological Control 47: 97-102 (2008)). The present disclosure provides that Cry proteins can be potential control agents of plant-parasitic nematodes in plants.

### Transgene Stacking

Methods for stacking transgenes are well known in the art. For example, the GAANTRY system (Gene Assembly in Agrobacterium by Nucleic acid Transfer using Recombinase technologY) leverages recombinase-mediated stacking technology. The specificity and efficiency of recombinases make them extremely attractive for genome engineering. Advancements in molecular biology and recombinases have paved the way for gene stacking with the assistance of unidirectional recombination systems. Development of this high-efficiency gene stacking system uses the specificity of the recombinases to effectively deliver the target genes of interest to a predetermined position. This is a flexible and effective system for stably stacking multiple genes within an Agrobacterium virulence plasmid Transfer-DNA (T-DNA).

Agrobacterium-mediated transformation of plants with one or a few genes is relatively routine, but the assembly and transformation of large constructs carrying multiple genes and their efficient use to generate high-quality transgenic plants has been a challenge.

The present disclosure teaches GAANTRY, which is a flexible and effective system for stably stacking multiple genes within an Agrobacterium virulence plasmid Transfer-DNA (T-DNA). This GAANTRY system is well described in Collier, R. et al (2018), Plant Journal 95, 573-583 and McCue et al (2019) BMC Research Notes 12, 457, each of which is incorporated herein by reference for all purposes.

The GAANTRY system is based on the combined use of unidirectional integration and excision controlled by three site-specific serine recombinases, which is an effective and stable system for stacking multiple genes within an Agrobacterium virulence plasmid T-DNA. The gene stacking system utilizes ‘P-Donor’ and ‘B-Donor’ cloning vectors, and ‘P-Helper’ and ‘B-Helper’ vectors, for the insertion of sequences of interest. The P-Donor and B-Donor vectors contain either attP or attB, respectively, recognition sites enabling precise integration into the virulence plasmid of the GAANTRY ArPORT1 strain. Plant transformation with T-DNA expressed from a GAANTRY modified Agrobacterium can produce high quality events that contain low copies of a complete T-DNA with limited incorporation of vector ‘backbone’ sequences (Collier, 2018).

The resulting engineered Agrobacterium strain can be directly used for plant transformation. The gene stacking strategy is efficient, precise, modular, and allows control over the orientation and order in which genes are stacked within the T-DNA.

In some embodiments, at least 2, at least 3, at least 4, at least 5, at least 6, at least 7, at least 8, at least 9, or at least 10 T-DNAs can be stacked in the DNA constructs taught herein.

Many traits have been identified that are not regularly selected for in the development of a new variety but that can be improved by backcrossing and genetic engineering techniques. These traits may or may not be transgenic; examples of these traits include but are not limited to: herbicide resistance; resistance to bacterial, fungal or viral disease; insect resistance; uniformity or increase in concentration of starch and other carbohydrates; enhanced nutritional quality; decrease in tendency of tuber to bruise; and decrease in the rate of starch conversion to sugars. These genes are generally inherited through the nucleus.

### Plants for Use with the Disclosed Methods

### Taxonomy of the Genus Solanum

The Solanaceae family contains several well-known cultivated crops such as tomato (Solanum lycopersicum also referred to as Lycopersicon esculentum), eggplant (Solanum melogena), tobacco (Nicotiana tabacum), pepper (Capsicum annuum) and potato (Solanum tuberosum). Within the genus Solanum, over a thousand species have been recognized. Potatoes will not hybridize with non-tuber bearing Solanum (tomato, eggplant, etc.) species including weeds commonly found in and around commercial potato fields (Love 1994).

The genus Solanum is divided into several subsections, of which the subsection potato contains all tuber-bearing potatoes. The subsection potato is divided into series, of which tuberosa is relevant to this document. Within the series tuberosa approximately 54 species of wild and cultivated potatoes are found. One of these is S. tuberosum.

S. tuberosum is divided into two subspecies: tuberosum and andigena. The subspecies tuberosum is the cultivated potato widely in use as a crop plant in, for example, North America and Europe. The subspecies andigena is also a cultivated species, but cultivation is restricted to Central and South America (Hanneman 1994). Z. Huamán and D. M. Spooner reclassified all cultivated potatoes as a single species, S. tuberosum, with various groups, including a Tuberosum Group (S. tuberosum subsp. Tuberosum) for the modern cultivars (Huamán et al., Am. J. of Botany 89(6): 947-965. 2002)

The only two wild potato species that grow within the borders of the USA, and for which specimens exist in gene banks, include the tetraploid species S. fendleri (recently reclassified as S. stoloniferum; however, some sources, including the Inter-genebank Potato Database, still use the S. fendleri designation) and the diploid species S. jamesii (Bamberg et al. 2003; IPD 2011; Bamberg and del Rio 2011a; Bamberg and del Rio 2011b; Spooner et al. 2004). Love (1994) reported that a third species, S. pinnatisectum, is also a native species in the USA. However, Spooner et al. (2004) determined that what was previously thought to be S. pinnatisectum was in fact S. jamesii. Through more than 10 years of field work and assessments of existing records, Bamberg et al. (2003) and Spooner et al. (2004) established the presence of only these two species, S. fendleri and S. jamesii, in the U.S. These researchers also attempted to verify previously recorded locations, and through this process, updated the maps of current known locations of these species, providing latitude and longitude locations for each documented population (Bamberg et al. 2003) and distribution maps (Spooner et al. 2004). These species mostly reside in dry forests, scrub desert, and sandy areas at altitudes of 5,000 to 10,000 feet, well isolated from most commercial production areas (Bamberg and del Rio 2011a).

While there is some overlap between the acreage used for commercial production and occurrence of wild species on a county level, the majority of the potato production in the United States is not in wild potato zones. However, there is a possibility that a few wild potato plants may be growing near potato fields (Love 1994). Spooner et al. (2004) describe S. jamesii habitat in the U.S. as among boulders on hillsides, sandy alluvial stream bottoms, in gravel along trails or roadways, rich organic soil of alluvial valleys, sandy fallow fields, grasslands, juniper-pinyon scrub deserts, oak thicket, coniferous and deciduous forests at elevations between 4,500 to 9,400 feet. They describe S. fendleri habitat similarly, and at elevations between 4700 to 11,200 feet.

With respect to potato plants, Solanum tuberosum subsp. tuberosum is an example of one of the most widely cultivated potato varieties, although there are thousands of potato varieties worldwide. Examples of well-known cultivated varieties that may be used with the methods and sequences disclosed herein include, but are not limited to, russets, reds, whites, yellows (also called Yukons) purples, Adirondack Blue, Adirondack Red, Agata, Almond, Amandine, Anya, Arran Victory, Atlantic, Bamberg, Belle de Fontenay, BF-15, Bildtstar, Bintje, Blue Congo, Bonnotte, Cabritas, Camota, Chelina, Chiloé, Cielo, Clavela Blanca, Désirée, Fianna, Fingerling, Flava, Golden Wonder, Innovator, Jersey Royal, Kerr's Pink, Kestrel, King Edward, Kipfler, Lady Balfour, Linda, Marfona, Maris Piper, Marquis, Nicola, Pachacoñ a, Pink Eye, Pink Fir Apple, Primura, Ratte, Red Norland, Red Pontiac, Rooster, Russet Burbank, Russet Norkotah, Selma, Shepody, Sieglinde, Sirco, Spunta, Stobrawa, Vivaldi, Vitelotte, Yellow Finn, and Yukon Gold. Any of these cultivated varieties may be transformed or modified as disclosed herein to positively or negatively express target gene(s) taught herein for conferring resistance or tolerance to a fungal pathogen.

In some embodiments, the methods of the present disclosure confer resistance to a fungal pathogen in a Solanum tuberosum plant. In some embodiments, the S. tuberosum plant is resistant to at least one of the following fungal pathogens: Alternaria spp., Colletotrichum coccodes, Rhizoctonia solani Fusarium spp., Phoma spp., Helminthosporium solani, Polyscytalum pustulans, Verticillium spp., Helicobasidium mompa, and Synchytrium endobioticum. In some embodiments, the fungal pathogen is Alternaria alternata or Alternaria solani.

In some embodiments, the disclosure relates to a S. tuberosum plant produced by the methods disclosed herein, wherein the plant is resistant Alternaria alternata or Alternaria solani.

In some embodiments, the disclosure relates to a Solanum tuberosum plant, or part thereof, comprising a nucleic acid encoding an amino acid sequence at least 90% identical to SEQ ID NO: 1. In some embodiments, the nucleic acid is SEQ ID NO: 3, or a sequence at least 75% identical thereto. In some embodiments, the Solanum tuberosum plant, or part thereof, further comprises ScGAME17 and/or ScGAME18. In some embodiments, the Solanum tuberosum plant, or part thereof, has been modified to reduce expression of Sgt3 and/or block activity of (3-steroidal glycoalkaloid rhamnosyltransferase. In some embodiments, the Solanum tuberosum plant, or part thereof, further comprises an Sgt3 antisense transgene.

In some embodiments, the disclosure relates to a Solanum tuberosum plant, or part thereof, comprising a nucleic acid encoding an amino acid sequence at least 90% identical to SEQ ID NO: 2. In some embodiments, the nucleic acid is SEQ ID NO: 4, or a sequence at least 75% identical thereto. In some embodiments, the Solanum tuberosum plant, or part thereof, further comprises ScGAME17 and/or ScGAME18. In some embodiments, the Solanum tuberosum plant, or part thereof, has been modified to reduce expression of Sgt3 and/or block activity of (3-steroidal glycoalkaloid rhamnosyltransferase. In some embodiments, the Solanum tuberosum plant, or part thereof, further comprises an Sgt3 antisense transgene.

In some embodiments, the Solanum tuberosum plant or plant part is Solanum tuberosum sub sp. tuberosum.

### Plant Breeding

The disclosure has use over a broad range of plants, monocots and dicots, including species from the genera Asparagus, Atropa, Avena, Brassica, Citrus, Citrullus, Capsicum, Cucumis, Cucurbita, Daucus, Fragaria, Glycine, Gossypium, Helianthus, Heterocallis, Hordeum, Hyoscyamus, Lactuca, Linum, Lolium, Lycopersicon, Malus, Manihot, Majorana, Medicago, Nicotiana, Oryza, Panieum, Pannesetum, Persea, Pisum, Pyrus, Prunus, Raphanus, Secale, Senecio, Sinapis, Solanum, Sorghum, Trigonella, Triticum, Vitis, Vigna, and Zea. Examples include tobacco and Arabidopsis, cereal crops such as maize, wheat, rice, soybean barley, rye, oats, sorghum, alfalfa, clover and the like, oil-producing plants such as canola, safflower, sunflower, peanut and the like, vegetable crops such as tomato tomatillo, potato, pepper, eggplant, sugar beet, carrot, cucumber, lettuce, pea and the like, horticultural plants such as aster, begonia, chrysanthemum, delphinium, zinnia, lawn and turfgrasses and the like.

The research leading to potato varieties which combine the advantageous characteristics referred to above is largely empirical. This research requires large investments of time, labor, and money. The development of a potato cultivar can often take a long time (up to eight years or more) from greenhouse to commercial usage. Breeding begins with careful selection of superior parents to incorporate the most important characteristics into the progeny. Since all desired traits usually do not appear with just one cross, breeding must be cumulative.

Present breeding techniques continue with the controlled pollination of parental clones. Typically, pollen is collected in gelatin capsules for later use in pollinating the female parents. Hybrid seeds are sown in greenhouses and tubers are harvested and retained from thousands of individual seedlings. The next year one to four tubers from each resulting seedling are planted in the field, where extreme caution is exercised to avoid the spread of virus and diseases. From this first-year seedling crop, several “seed” tubers from each hybrid individual which survived the selection process are retained for the next year's planting. After the second year, samples are taken for density measurements and fry tests to determine the suitability of the tubers for commercial usage. Plants which have survived the selection process to this point are then planted at an expanded volume the third year for a more comprehensive series of fry tests and density determinations. At the fourth-year stage of development, surviving selections are subjected to field trials in several states to determine their adaptability to different growing conditions. Eventually, the varieties having superior qualities are transferred to other farms and the seed increased to commercial scale. Generally, by this time, eight or more years of planting, harvesting and testing have been invested in attempting to develop the new and improved potato cultivars.

Backcrossing methods can be used with the present disclosure to improve or introduce a characteristic into the variety. The term “backcrossing” as used herein refers to the repeated crossing 1, 2, 3, 4, 5, 6, 7, 8, 9 or more times of a hybrid progeny back to the recurrent parents. The parental potato plant which contributes the gene(s) for the one or more desired characteristics is termed the nonrecurrent or donor parent. This terminology refers to the fact that the nonrecurrent parent is used one time in the backcross protocol and therefore does not recur. The parental potato plant to which the gene or genes from the nonrecurrent parent are transferred is known as the recurrent parent as it is used for several rounds in the backcrossing protocol. In a typical backcross protocol, the original variety of interest (recurrent parent) is crossed to a second variety (nonrecurrent parent) that carries the gene(s) of interest to be transferred. The resulting progeny from this cross are then crossed again to the recurrent parent and the process is repeated until a potato plant is obtained wherein essentially all of the desired morphological and physiological characteristics of the recurrent parent are recovered in the converted plant, in addition to the one or more genes transferred from the nonrecurrent parent.

The selection of a suitable recurrent parent is an important step for a successful backcrossing procedure. The goal of a backcross protocol is to alter or substitute one or more traits or characteristics in the original variety. To accomplish this, one or more genes of the recurrent variety are modified, substituted or supplemented with the desired gene(s) from the nonrecurrent parent, while retaining essentially all of the rest of the desired genes, and therefore the desired physiological and morphological constitution of the original variety. The choice of the particular nonrecurrent parent will depend on the purpose of the backcross. One of the major purposes is to add some commercially desirable, agronomically important trait to the plant. The exact backcrossing protocol will depend on the characteristic or trait being altered or added to determine an appropriate testing protocol. Although backcrossing methods are simplified when the characteristic being transferred is a dominant allele, a recessive allele may also be transferred. In this instance, it may be necessary to introduce a test of the progeny to determine if the desired characteristic has been successfully transferred.

### Molecular Techniques

With the advent of molecular biological techniques that have allowed the isolation and characterization of genes that encode specific protein products, scientists in the field of plant biology developed a strong interest in engineering the genome of plants to contain and express foreign genes, or additional, or modified versions of native, or endogenous, genes (perhaps driven by different promoters) in order to alter the traits of a plant in a specific manner. Such foreign additional and/or modified genes are referred to herein collectively as “transgenes”. Over the last fifteen to twenty years several methods for producing transgenic plants have been developed, and the present disclosure, in particular embodiments, also relates to transformed versions of the claimed variety or line.

Plant transformation involves the construction of an expression vector which will function in plant cells. Such a vector comprises DNA comprising a gene under control of, or operatively linked to, a regulatory element (for example, a promoter). The expression vector may contain one or more such operably linked gene/regulatory element combinations. The vector(s) may be in the form of a plasmid, and can be used alone or in combination with other plasmids, to provide transformed plants, using transformation methods as described below to incorporate transgenes into the genetic material of the plant(s).

Traditional plant breeding typically relies on the random recombination of plant chromosomes to create varieties that have new and improved characteristics. According to standard, well-known techniques, genetic “expression cassettes,” comprising genes and regulatory elements, are inserted within the borders of Agrobacterium-isolated transfer DNAs (“T-DNAs”) and integrated into plant genomes. Agrobacterium-mediated transfer of T-DNA material typically comprises the following standard procedures: (1) in vitro recombination of genetic elements, to produce an expression cassette for selection of transformation, (2) insertion of this expression cassette, often together with at least one other expression cassette into a T-DNA region of a binary vector, (3) transfer of the sequences located between the T-DNA borders, often accompanied with some or all of the additional binary vector sequences from Agrobacterium to the plant cell, and (4) selection of stably transformed plant cells that display a desired trait, such as an increase in yield, improved vigor, enhanced resistance to diseases and insects, or greater ability to survive under stress.

In some embodiments, genetic engineering methods may rely on the introduction of foreign, not-endogenous nucleic acids, including regulatory elements such as promoters and terminators, and genes that are involved in the expression of a new trait or function as markers for identification and selection of transformants, from viruses, bacteria and plants. Marker genes are typically derived from bacterial sources and confer antibiotic or herbicide resistance. In the “anti-sense” technology, the sequence of native genes is inverted to silence the expression of the gene in transgenic plants.

### Compositions for Controlling Pest and Pathogens

In some embodiments, the disclosure relates to compositions comprising an effective amount of isolated tetraose glycoalkaloids (amount of resistance being proportional to the amount of tetraose glycoalkaloid); and an adjuvant. In some embodiments, the tetraose glycoalkaloids are selected from the group consisting of Solanidine-Gal-Glu-Glu-Glu, Solanidine-Gal-Glu-Glu-Xyl, and Solanidine-Gal-Glu-Rha-Glu. In some embodiments, the composition is a fungicide. In some embodiments, the composition is a broad-spectrum fungicide. In some embodiments, the composition is a pesticide.

The compositions of the disclosure offer several advantages over currently used fungicides. First, the active ingredient are naturally-occurring isolated tetraose glycoalkaloids which may be synthetically produced or derived from plant extracts. In some embodiments, the disclosure provides methods of controlling fungal plant pathogens or fungal-like pathogens comprising contacting the vegetation or an area adjacent thereto to prevent the growth of a fungal or fungal-like pathogen with an effective amount of the compositions disclosed herein.

The composition may be solid (i.e., in a powdered form) or liquid depending on the carrier and the needs of the agriculturist using the composition. If the composition is solid, suitable carriers include various known, agriculturally-useful powders that are generally used for this purpose. If the composition is liquid, it may be aqueous or non-aqueous and may be a solution, suspension, or emulsion, depending on the needs of the agriculturist applying the composition. It is also contemplated that a liquid composition may be comprised solely of the active ingredient of tetraose glycoalkaloids.

In some embodiments, the composition may be prepared as a concentrate for industrial application and further dilution or as a fully diluted ready-to-apply composition. The composition may comprise a surfactant carrier to effect miscibility or suspendability of the composition in a liquid.

In some embodiments, the subject composition can include an antioxidant at a level sufficient to increase the product shelf life, inhibit decomposition of the active ingredient, or improve the stability of the controlling effect when the composition is applied to hosts infested with fungal or fungal-like pathogen. Example antioxidants, include, but are not limited to, ascorbyl palmitate, anoxomer, benzoic acid, benzlkonium chloride, benzethonium chloride, benzyl alcohol, butylated hydroxyanisole, butylated hydroxytoluene, chlorobutanol, dehydroacetic acid, ethylenediamine, ferulic acid, potassium benzoate, potassium metabisulfite, potassium sorbate, n-propyl gallate BP, propylparaben, sassafras oil, sodium benzoate, sodium bisulfite, sodium metabisulfite, sorbic acid, vitamin E, eugenol, and the like.

In some embodiments, the composition can be an aqueous composition using water as the solvent or an organic composition using an organic solvent. In some embodiments, the composition contains one or more surface-active agents in amounts sufficient to render a given composition readily dispersible in water or in an organic solvent.

By the term “surfactant” it is understood that wetting agents, surface-active agents or surfactants, dispersing agents, suspending agents, emulsifying agents, and combinations thereof, are included therein. Ionic and non-ionic surface-active agents can be used.

Nonionic surfactants are comprised of linear or nonyl-phenol alcohols and/or fatty acids. This class of surfactant reduces surface tension and improves spreading, sticking and herbicide uptake. Examples of non-ionic surface-active agents include, but are not limited to, alkoxylates, N-substituted fatty acid amides, amine oxides, esters, sugar-based surfactants, polymeric surfactants, and mixtures thereof, allinol, nonoxynol, octoxynol, oxycastrol, oxysorbic (for example, polyoxyethylated sorbitol fatty-acid esters (TWEEN®), thalestol, and polyethylene glycol octylphenol ether (TRITON®).

Examples of ionic surfactants for use with the fungicidal composition described herein may include anionic surfac-tants such as alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignin sul-fonates, sulfonates of fatty acids and oils, sulfonates of ethoxylated alkylphenols, sulfonates of alkoxylated arylphe-nols, sulfonates of condensed naphthalenes, sulfonates of dodecyl- and tridecylbenzenes, sulfonates of naphthalenes and alkylnaphthalenes, sulfosuccinates or sulfosuccina-mates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl car-boxylates, and carboxylated alcohol or alkylphenol ethoxy-lates.

Usually, the amount of surfactant used is the minimum amount required to get the compound into solution/emulsion, and will generally be 0.1 to 10% by weight.

The common and chemical names of other generally available adjuvants include, but are not limited to, the following list, in which the first name is the common name used in the industry, the second name is the general chemical name, the third name is the class of the compound, the fourth name is the type of surfactant, and the trade name is last.

Albenate: Alkyl(C₁₈ C₂₄) benzene sulfonic acid and its salts; Alkylaryl sulfonate; Anionic surfactant; Nacconol 88SA, Calsoft F-90, DDBSA, Santomerse No. 3.

Alfos: α-Alkyl(C₁₀-C₁₆)-ω-hydroxypoly(oxyethylene) mixture of dihydrogen phosphates esters; polyoxyethylene alkyl phosphate ester; Anionic; Emcol PS-131.

Allinate: αa-Lauryl-ω-hydroxypoly(oxyethylene) sulfate; lauryl polyoxyethylene sulfate salts; Anionic; Sipon ES.

Allinol: α-Alkyl(C₁₁-C₁₅)-ω-hydroxypoly(oxyethylene); C₁₁C₁₅ linear primary alcohol ethoxylate; Nonionic; Neodol 25-3, Alfonic 1014-40 and other alfonic materials.

Diocusate: Sodium dioctyl-sulfosuccinate; Dioctyl sodium sulfosuccinate; Anionic; TRITON GR-5, Aerosol OT.

Dooxynol: α-(p-Dodecyl-phenyl)-ω-hydroxypoly(oxyethylene); dodecylphenol condensation with ethylene oxide; Nonionic; Igepal RC-630, Tergitol 12-P-9, Sterox D Series.

Ligsolate: Lignosulfonate, NH₄, Ca, Mg, K, Na, and Zn salts; Salts of lignosulfonic acids; Anionic; Marasperse N-22, Polyfon O.

Nofenate: α-(p-Nonylphenyl)-ω-hydroxypoly(oxyethelene) sulfate, NH₄, Ca, Mg, K, Na, Zn salts, Nonyl group is a propylene trimer isomer; Salts of sulfate ester of nonylphynoxypoly(ethyleneoxy) ethanol; Anionic; Alipal CO Series.

Nonfoster: α-(p-Nonylphenyl)-ω-hydroxypoly(oxyethylene); mixture of dihydrogen phosphate and nonophosphate esters; Polyoxyethylene nonylphenol phosphate esters; Anionic; Gafac RM 510.

Nonoxynol: α-(p-Nonylphenyl)-ω-hydroxypoly(oxyethylene); polyoxyalkylene nonylphenol; Nonionic; Sterox N Series, Makon 6, Igepal CO Series TRITON N Series, T-DET N.

Octoxynol: α-[p-1,1,3,3 -Tetramethyl butyl phenyl]-ω-hydroxypoly(oxyethylene); polyoxyethylene octyl phenol; Nonionic; Igepal CA-630, TRITON X-100.

Oxycastol: Castor oil polyoxyethylated; Ethoxylated castor oil; Nonionic; Emulphor EL-719, Emulphor EL-620, Trylox CO-40, T-DET C-40

Oxysorbic: Polyoxyethylated sorbitol fatty acid esters (nonosterate, monoleate etc); Polyoxyethylated sorbitol fatty acid esters; Nonionic; Atlox 1045, Drewmulse POE-STS, TWEEN Series G-1045.

Tall oil: Tall oil, fatty acids not less than 58%, rosin acids not greater than 44%, unsapolifiables not greater than 8%; Tall oil; Anionic; Ariz. S. A. Agent 305.

Thalestol: Polyglyceryl phthalate ester of coconut oil fatty acid; Modified phthalic glycerol alkyl resin; Nonionic; TRITON B-1956.

The composition may include other active or inactive ingredients. In some embodiments, the composition includes at least one additional fungicide. Example additional fungicides include, but are not limited to, azoxystrobin, bifujunzhi, coumethoxystrobin, coumoxystrobin; dimoxystrobin, enes-troburin, enoxastrobin, fenaminstrobin, fenoxystrobin, flufenoxystrobin, fluoxastrobin, jiaxiangjunzhi, kresoxim-methyl, mandestrobin, metominostrobin, orysastrobin, picoxystrobin, pyraclostrobin, pyrametostrobin, pyraox-ystrobin, triclopyricarb, trifloxystrobin, methyl 2-[2-(2,5-dimethy 1phenyloxymethy 1)pheny 1]-3-methoxyacry late, pyribencarb, triclopyricarb/chlorodincarb, famoxadon, fena-midon, cyazofamid, amisulbrom, benodanil, bixafen, boscalid, carboxin, fenfuram, fluopyram, flutolanil, fluxapy-roxad, furametpyr, isopyrazam, mepronil, oxycarboxin, pen-flufen, penthiopyrad, sedaxane, tecloftalam, thifluzamide, N-(4′-trifluoromethylthio-bipheny 1-2-yl)-3-difluoromethy 1-1-methy 1-1 H-pyrazole-4-carboxamide, N-(2-(1,3,3-trimeth-ylbutyl)phenyl)-1,3-dimethyl-5-fluoro-1 H-pyrazole-4-car-boxamide, N-[9-(dichloromethylene)-1,2,3,4-tetrahydro-1,4-methanonaphthalen-5-yl]-3-(difluoromethyl)-1-methyl- H-pyrazole-4-carboxamide, diflumetorim, binapacryl, dinobuton, dinocap, meptyl-dinocap, fluazinam, ferimzone, ametoctradin, silthiofam, azaconazole, bitertanol, bromuconazole, cyproconazole, difenoconazole, diniconazole, diniconazole-M, epoxiconazole; fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, imazalil, pefurazoate, prochloraz, triflumizole, pyrimidines, fenari-mol, nuarimol, pyrifenox, triforine, aldimorph, dodemorph, dodemorph acetate, fenpropimorph, tridemorph, fenpropidin, piperalin, spiroxamine, fenhexamid, benalaxyl, benal- axyl-M, kiralaxyl; metalaxyl, metalaxyl-M (mefenoxam), ofurace; oxadixyl, hymexazole, octhilinone, oxolinic acid, bupirimate, benomyl, carbendazim, fuberidazole, thiaben-dazole, thiophanate-methyl, 5-chloro-7-(4-methyl-piperi-din-1-yl)-6-(2,4,6-trifluorophenyl)-[1,2,4]triazolo[1,5-a]pyrimidine, diethofencarb, ethaboxam, pencycuron, fluopicolid, zoxainid, metrafenon, pyriofenon, cyprodinil, mepanipyrim, pyrimethanil, fluoroimide, iprodione, procymidone, vinclozolin fenpiclonil, fludioxonil, quinoxyfen, edifenphos, iprobenfos, pyrazophos, isoprothiolane, dicloran, quintozene, tecnazene, tolclofos-methyl, biphenyl, chloroneb, etridiazole, dimethomorph, flumorph, mandipropamid, pyrimorph, benthiavalicarb, iprovalicarb, valifenal-ate and 4-fluorophenyl N-(1-(1-(4-cyanophenyl)ethanesul-fonyl)but-2-yl)carbamate, propamocarb, propamocarb hydrochloride, ferbam, mancozeb, maneb, metiram, propineb, thiram, zineb, ziram, anilazine, chlorothalonil, captafol, captan, folpet, dichlofluanid, dichlorophen, flusulfamide, hexachlorobenzene, pentachlorophenol, phthalid, tolylfluanid, N-(4-chloro-2-nitrophenyl)-N-ethyl-4-methyl-benzenesulfonamide, guanidine, dithianon, validamycin, polyoxin B, pyroquilon, tricyclazole, carpropamid, dicyclomet, fenoxanil, and mixtures thereof.

In some embodiments, the composition further comprises an additional compound selected from the group consisting of azoxystrobin, bifujunzhi, coumethoxystrobin, coumoxystrobin, dimoxystrobin, enestroburin, enoxastrobin, fenaminstrobin, fenoxystrobin, flufenoxystrobin, fluoxastrobin, jiaxiangjunzhi, kresoxim-methyl, mandestrobin, metominostrobin, orysastrobin, picoxystrobin, pyraclostrobin, pyrametostrobin, pyraoxystrobin, triclopyricarb, trifloxystrobin, methyl 2-[2-(2,5-dimethylphenyloxymethyl)phenyl]-3-methoxyacryl ate, azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, imazalil, pefurazoate, prochloraz, triflumizole, pyrimidines, fenarimol, nuarimol, pyrifenox, and triforine.

In some embodiments, the adjuvant of the composition is a surfactant. In some embodiments, the surfactant is a non-ionic surfactant, crop oil concentrate, nitrogen-surfactant blend, esterified seed oil, or an organo-silicone.

In some embodiments, nonionic surfactants are comprised of linear or nonyl-phenol alcohols and/or fatty acids. This class of surfactant reduces surface tension and improves spreading, sticking and composition uptake.

In some embodiments, crop oil concentrates are composed of a blend of paraffinic-based petroleum oil and surfactants. This surfactant class reduces surface tension and improves composition uptake and target surface spreading.

In some embodiments, nitrogen-surfactant blends consist of premix combinations of various forms of nitrogen and surfactants. These surfactants reduce surface tension and improve target surface spreading.

In some embodiments, esterified seed oils are produced by reacting fatty acids from seed oils (corn, soybean, sunflower, canola) with an alcohol to form esters. The methyl or ethyl esters produced by this reaction are combined with surfactants/emulsifiers to form an esterified seed oil. These surfactants reduce surface tension and improve composition uptake by improving composition distribution on the target surface.

In some embodiments, organo-silicones are usually silicone/surfactant blends of silicone to nonionic or other surfactants; a few within this classification are composed entirely of silicone. These surfactants provide a tremendous reduction in surface tension and spread more than conventional surfactants.

In general, three types of oils are commonly referred to as surfactants: vegetable seed oils, crop oil concentrates, and esterified seed oils. First, vegetable seed oils are a blend of vegetable oil (cottonseed, soybean) and surfactants. These surfactants exhibit good crop tolerance but do not have good spreading, sticking or pest-penetrating properties. Second, crop oil concentrates are a blend of paraffinic oil (petroleum based) and surfactants. These surfactants exhibit good spreading and penetrating properties. Third, esterified seed oils are comprised of a methyl or ethyl ester of a vegetable seed oil (sunflower, soybean, corn, canola) combined with a surfactant/emulsifier. These spray solution additives have good spreading and pest-penetrating properties and convey good crop tolerance.

In some embodiments, the composition is a powder, granule, or liquid.

### EXAMPLES

The foregoing examples of the related art and limitations related therewith are intended to be illustrative and not exclusive. Other limitations of the related art will become apparent to those of skill in the art upon a reading of the specification.

### Example 1: Generating Plants with Resistance Gene(s) ScGTR1 and ScGTR2

As described above in the detailed description, transient expression of ScGTR1 and ScGTR2 conveyed resistance to susceptible S. commersonii line CGN18024_3. Briefly, expression vectors comprising ScGTR1 and ScGTR2 were infiltrated into the leaves of CGN18024_1 (resistant S. commersonii), CGN18024_3 (susceptible S. commersonii), Atlantic and Bintje potatoes (both susceptible S. tuberosum). The infiltrated spots were inoculated with spores from A. solani two days after agroinfiltration. Sizes of the lesions were measured 5 days post inoculation as a measure for disease resistance. As shown in FIG. 3A, CGN18024 1 remained resistant in all cases, while large lesions developed on leaves of CGN18024_3 where it was infiltrated with an empty vector (‘-’) or the (non-functional) GT allele (ScGTS2) from the susceptible haplotype (‘S2’). Lesions on the spots where ScGTR1 and ScGTR2 were expressed (‘R1’ and ‘R2’) were much smaller compared to these controls (FIG. 3A & FIG. 3B), showing that expression of ScGTR1 and ScGTR2 in an otherwise susceptible S. commersonii line can indeed convey resistance to A. solani. However, there was no clear difference between the spots that were infiltrated in Atlantic or Bintje (FIG. 3C & FIG. 3D).

To better study the effect of ScGTR1 and ScGTR2 on resistance of S. commersonii, and possibly S. tuberosum to fungal pathogens including Alternaria genus, two susceptible potato plants (S. commersonii 18024_3 and Atlantic) were transformed with overexpression constructs for both genes. Greater than 40 transformants of the stable transgenics were obtained for each transformation. A disease screen was performed on the transformants to evaluate resistance to A. solani.

Similar to the results seen in the transient assay, all Atlantic transformants appeared susceptible to A. solani despite presence of ScGTR1 or ScGTR2, whereas almost all 18024_3 transformants were found to be resistant. Just one out of thirty seven 18024_3 transformants with ScGTR1 was found to be susceptible and only 2 out of thirty three 18024_3 transformants with ScGTR2 displayed susceptibility. This experiment confirms the results that were described above in the transient assays.

The experiments described above indicate that expression of either ScGTR1 or ScGTR2 can confer resistance to S. commersonii, but not to S. tuberosum. Additional data obtained from plant extracts and metabolite analysis (described above in the detailed description and below in Example 2) show that resistance is conferred by a host-specific compound, specifically, tetraose glycoalkaloids.

### Example 2: Compositions for Pest and Pathogen Control

As described above in the detailed description section, leaf extracts from resistant line CGN18024-1 strongly inhibited growth of A. solani at concentrations ≥2.5% (FIG. 4A) and this was not affected by autoclaving. Additionally, a few days after A. solani was plated, fungal contamination appeared on the plates containing semi-sterilized leaf extract from CGN18024_3. Remarkably, such contamination did not appear on the plates containing extract of CGN18024_1 (FIG. 4B). Thus, it appears that the compound(s) from CGN18024_1 not only inhibits growth of A. solani, but growth of other fungi as well.

To test this hypothesis, a similar experiment was performed with autoclaved extract from CGN18024_1 and CGN18024_3, respectively against another pathogen, Botrytis cinerea. It was confirmed that growth of B. cinerea is strongly inhibited by extract from CGN18024_1 and not by extract from CGN18024_3 (FIG. 4D).

As described above in the detailed description, metabolite analysis showed that all resistant genotypes contained tetraose glycoalkaloids (composed of an aglycone conjugated to an oligosaccharide containing 4 monosaccharide moieties), whereas susceptible genotypes contained only triose glycoalkaloids (containing 3 monosaccharide moieties instead of 4), or very low levels of tetraose glycoalkaloids (FIG. 5). Two distinct tetraoses were identified on glycoalkaloids of CGN18024_1: (1) Gal-Glu-Glu-Glu and (2) Gal-Glu-Glu-Xyl. In the transformation experiments described above and in Example 1, the ScGTR1 S. commersonii transformants contained Gal-Glu-Glu-Glu-alkaloids, whereas the ScGTR2 transformants contained almost exclusively Gal-Glu-Glu-Xyl alkaloids (FIG. 5). Thus, these data indicate that ScGTR1 is responsible for the glucosylation of a triose glycoalkaloid precursor in S. commersonii, while ScGTR2 catalyses xylosilation of the same substrate. Both types of conversions are likely to confer resistance to A. solani in S. commersonii.

Glycoalkaloids have been shown to serve as a protective barrier to a broad range of pathogens and pests (Cárdenas et al. 2015), and the specific glycoalkaloids that are identified in S. commersonii have been described to also be effective against pest insects such as Colorado potato beetle as well (Sinden, Sanford et al. 1980).

Thus, the tetraose glycoalkoloids described herein, for example, Solanidine-Gal-Glu-Glu-Glu and Solanidine-Gal-Glu-Glu-Xyl (see also Table 2 and FIG. 6A-6C) can be used as compounds to exogenously control plant pests and pathogens, such as early blight. The tetraose glycoalkoloids may be applied to a plant, or soil surrounding a plant, for example as a foliar spray or root drench, or they may be in a composition comprising other ingredients, for example adjuvants, surfactants, and other fungicides.

Data also suggests that these tetraose glycoalkoloids and compositions comprising them may also be effective against other pests and pathogens such as Botrytis, Verticillium, Fusarium, Rhizoctonia, Insects (such as Colorado potato beetle), and Phytophthora.

### Example 3: Identification of GAME Homologs

Based on the results described above, the resistance of S. commersonii to A. solani is caused by modification of a glycoalkaloid. The addition of an extra glycose (glucose or xylose) converts it to a version that strongly inhibits A. solani as well as other fungi.

The biosynthesis pathway that leads to the production of glycoalkaloids in potato and tomato is rather conserved and well-characterized, and the genes involved in this pathway are syntenic (FIG. 6A-6C) See Cardenas 2015. Moreover, the glycoalkaloids from S. commersonii and S. tuberosum have identical precursors (i.e. the solanidine aglycone). Combined with the availability of the S. commersonii genome, it was hypothesized that the missing or non-functional gene in S. tuberosum was a GAME homolog.

As the precursor is already produced in S. tuberosum, it is possible to produce the S. commersonii tetraose glycoalkaloid compounds by making genetic modifications to an existing, non-functional GAME homolog, or by expressing the missing enzyme(s) in S. tuberosum.

Shown below in Table 3 is a list of GAME glycosyltransferases that could be co-expressed with ScGTR1 and/or ScGTR2 (nucleotides sequence encoding SEQ ID NO: 1 and/or SEQ ID NO: 2, or nucleotide sequence SEQ ID NO: 3 and/or SEQ ID NO: 4) in otherwise susceptible Solanum species. See also Itkin M, Rogachev I, Alkan N, et al. 2011. Glycoalkaloid metabolism 1 is required for steroidal alkaloid glycosylation and prevention of phytotoxicity in tomato. The Plant Cell 23, 4507-4525, and Itkin M., et al. 2013. Biosynthesis of antinutritional alkaloids in solanaceous crops is mediated by clustered genes. Science 341(6142); 175-9. The genes could be stacked together in one vector, or transformed by separate vectors.

TABLE 3
Tomato (SI) GAME homologs

| | Gene ID & | UniProt Accession or |
|---|---|---|
| Protein | alternate names | SEQ ID NO. |
| Tomatine galactoside | S1GAME18 (101254109; | A0A3Q7H9X4 |
| glucosyltransferase | Solyc07g043500.1) | |
| UDP-glucosyltransferase | ScGAME18 | SEQ ID NO: 10 |
| | | SEQ ID NO: 9 |
| Tomatidine 3-O- | GAME1 | E5L3R8 |
| galactosyltransferase | | |
| βi-tomatine | GAME2 | E5L3S0 |
| xylosyltransferase | | |
| UDP-glucosyltransferase | ScGAME17 | SEQ ID NO: 12 |
| | | SEQ ID NO: 11 |
| Tomatine galactoside | S1GAME17 | A0A3Q7HCG1 |
| glucosyltransferase | | |

To understand genetic and evolutionary relevance of glycosyltransferases (SlGAMEs, StSGTs, ScGAMEs and ScGTRs) in Solanum species, the potato (St) and tomato (Sl) glycosyltransferases involved in biosynthesis of glycoalkaloids (presented in FIG. 6A-6C) were analyzed with two glycosyltransferases (ScGTR1 and ScGTR2) derived from resistant S. commersonii. FIG. 7 shows the genetic lineage of the glycosyltransferase genes across Solanum species (e.g. S. lycopersicum, S. commersonii and S. tuberosum). As orthologs of the tomato GAME homologs (S1GAME1 and S1GAME2) are identified in S. tuberosum and/or S. commersonii, the corresponding orthologs (e.g. StSGT1 and ScGAME1 for S1GAME1; StSGT3 for S1GAME2) are presented (FIG. 7). However, functional orthologs of S1GAME17 and S1GAME18 were not identified or present in S. tuberosum.

### Example 4: Co-expression of ScGAME18 with ScGTR1 and/or ScGRT2

From the results and experiments described above, a new type of broad-spectrum disease control can be achieved in susceptible Solanum species, such as S. tuberosum. Transgenic plants were successfully produced using a combination of the ScGTR1 and/or ScGTR2 alleles with S. commersonii GAME18 (SEQ ID NO: 10 and SEQ ID NO: 9).

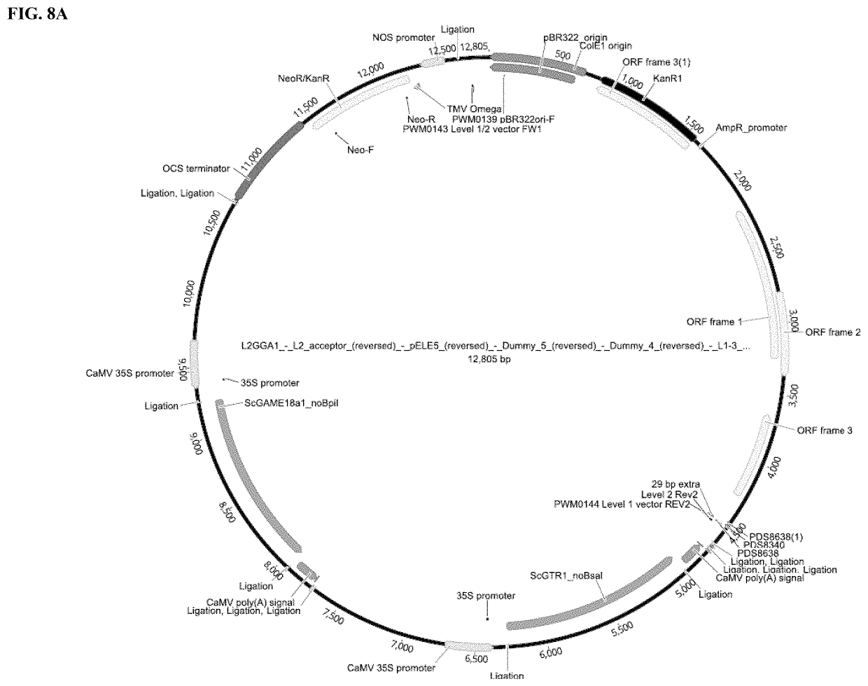

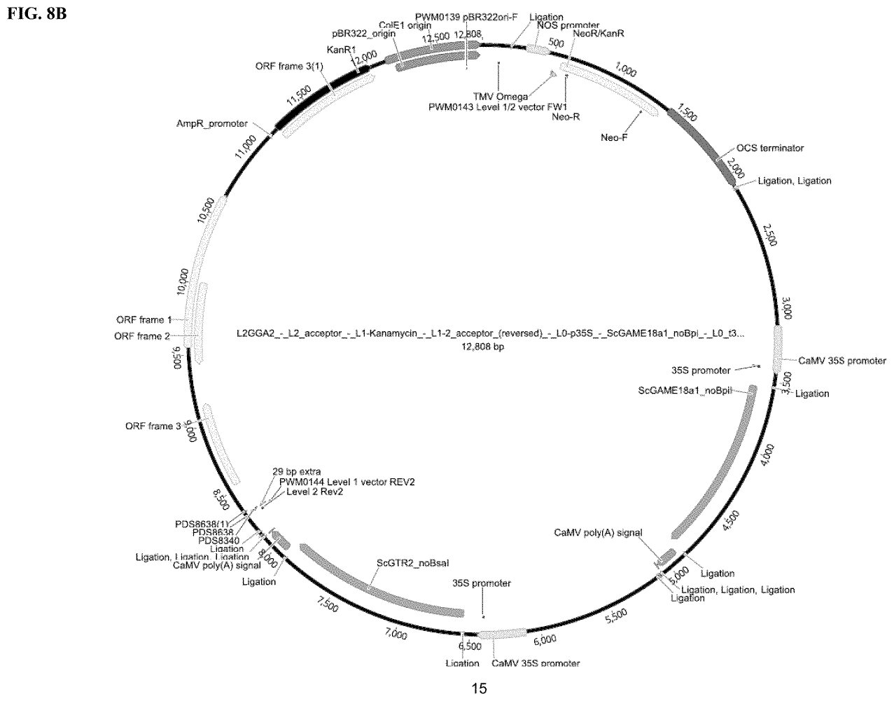

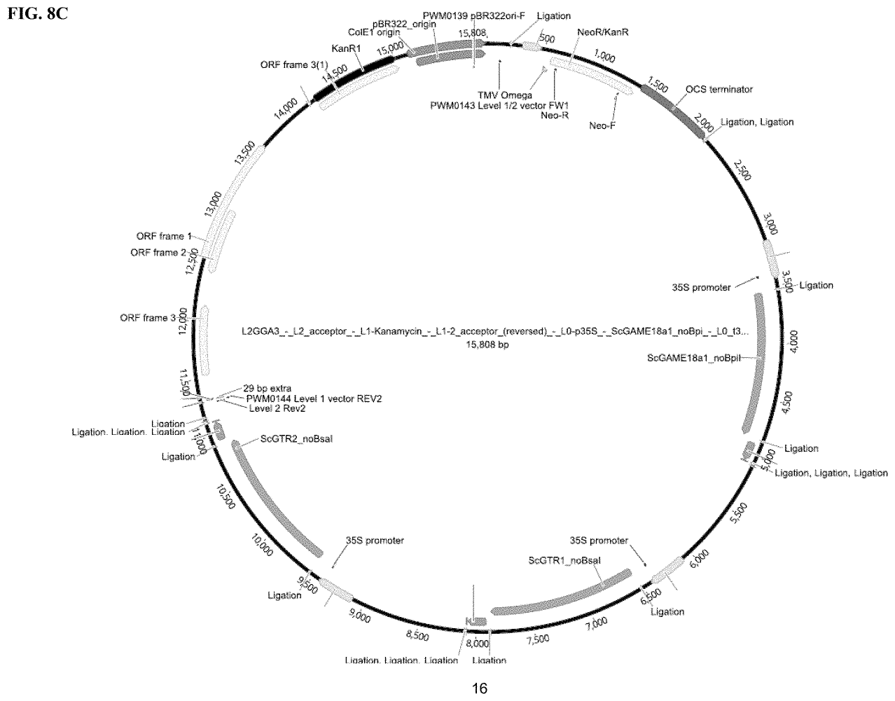

Three different constructs comprising 1) ScGAME18+ScGTR1 (FIG. 8A), 2) ScGAME18+ScGTR2 (FIG. 8B), and 3) ScGAME18+ScGTR1+ScGTR1 (FIG. 8C) were stabled transformed into Solanum tuberosum cv. Atlantic using standard plant transformation methods well known in the art. See, for example, Miki et al., “Procedures for Introducing Foreign DNA into Plants” in Methods in Plant Molecular Biology and Biotechnology, Glick, B. R. and Thompson, J. E. Eds. (CRC Press, Inc. Boca Raton, 1993) pages 67-88. In addition, expression vectors and in-vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pages 89-119.

It is expected that expression of these transgenes will produce tetraose glycoalkaloids such as Solanidine-Gal-Glu-Glu-Glu, Solanidine-Gal-Glu-Rha-Glu, and/or Solanidine-Gal-Glu-Glu-Xyl. This way, S. tuberosum can be engineered to have broad-spectrum resistance against a range of foliar microbial pathogens as well as pest insects.

### Deposit Information

Vector deposits of the J. R. Simplot Company proprietary vectors disclosed above and recited in the appended claims will be made with the American Type Culture Collection (ATCC), 10801 University Boulevard, Manassas, Va. 20110. The deposit of vectors will be taken from the same deposit maintained by J. R. Simplot Company since prior to the filing date of this application. All restrictions will be irrevocably removed upon granting of a patent, and the deposit is intended to meet all of the requirements of 37 C.F.R. §§ 1.801-1.809. The deposit will be maintained in the depository for a period of thirty years, or five years after the last request, or for the enforceable life of the patent, whichever is longer, and will be replaced as necessary during that period.

Tuber deposits of the J.R. Simplot Company proprietary Potato Cultivars/Events disclosed herewith will be made with an international depositary authority under the Budapest Treaty. The deposit will be taken from the same deposit maintained by J. R. Simplot Company. All restrictions will be irrevocably removed upon granting of a patent, and the deposit is intended to meet all of the requirements of 37 C.F.R. §§1.801-1.809. The deposit will be maintained in the depository for a period of thirty years, or five years after the last request, or for the enforceable life of the patent, whichever is longer, and will be replaced as necessary during that period.

### Incorporation by Reference

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

It should be understood that the above description is only representative of illustrative embodiments and examples. For the convenience of the reader, the above description has focused on a limited number of representative examples of all possible embodiments, examples that teach the principles of the disclosure. The description has not attempted to exhaustively enumerate all possible variations or even combinations of those variations described. That alternate embodiments may not have been presented for a specific portion of the disclosure, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the disclosure. Accordingly, the disclosure is not intended to be limited to less than the scope set forth in the following claims and equivalents.

### Numbered Embodiments of the Disclosure

Notwithstanding the appended claims, the disclosure sets forth the following numbered embodiments:
1. A method for conferring resistance or tolerance to a pest or pathogen in a Solanum spp. plant, plant part, or plant cell, comprising at least one of:
   a. introducing a nucleic acid into a Solanum spp. plant, plant part, or plant cell, wherein said nucleic acid encodes an amino acid sequence at least 90% identical to SEQ ID NO: 1, and
   b. introducing a nucleic acid into a Solanum spp. plant, plant part, or plant cell, wherein said nucleic acid encodes an amino acid sequence at least 90% identical to SEQ ID NO: 2.
2. The method of embodiment 1, wherein the nucleic acid of (a) comprises SEQ ID NO: 3, or a sequence at least 75% identical thereto.
3. The method of embodiment 1, wherein the nucleic acid of (b) comprises SEQ ID NO: 4, or a sequence at least 75% identical thereto.
4. The method of any one of the proceeding embodiments, further comprising introducing a nucleic acid encoding a functional homolog of a glycoalkaloid metabolism (GAME) glycosyltransferase polypeptide.
5. The method of embodiment 4, wherein the nucleic acid encodes a GAME18 and/or GAME17.
6. The method of embodiment 5, wherein the nucleic acid encodes an amino acid sequence at least 90% identical to SEQ ID NO: 10.
7. The method of embodiment 6, wherein the nucleic acid comprises SEQ ID NO: 9, or a sequence at least 75% identical thereto.
8. The method of embodiment 5, wherein the nucleic acid encodes an amino acid sequence at least 90% identical to SEQ ID NO: 12.
9. The method of embodiment 8, wherein the nucleic acid comprises SEQ ID NO: 11, or a sequence at least 75% identical thereto.
10. The method of any one of the proceeding embodiments, wherein expression of at least one of the nucleic acids generates tetraose glycoalkaloids, and wherein the tetraose glycoalkaloids confers resistance to a pest or pathogen.
11. The method of any one of the proceeding embodiment, wherein the Solanum spp. plant is resistant to at least one of the following fungal pathogens: Alternaria spp., Colletotrichum coccodes, Rhizoctonia solani Fusarium spp., Phoma spp., Helminthosporium solani, Polyscytalum pustulans, Verticillium spp., Helicobasidium mompa, and Synchytrium endobioticum. Botrytis cinerea, Sclerotina spp, Septoria spp.
12. The method of embodiment 11, wherein the fungal pathogen is Alternaria alternata or Alternaria solani.
13. The method of any one of the proceeding embodiments, wherein the Solanum spp. plant, plant part, or plant cell is S. tuberosum, S. melongena, or S. lycopersicum.
14. A Solanum spp. plant produced by the method of any one of embodiments 1-13.
15. A method for conferring resistance to a fungal pathogen in a Solanum tuberosum plant, plant part, or plant cell, comprising:
   a) introducing a nucleic acid into a S. tuberosum plant, plant part, or plant cell, wherein said nucleic acid encodes an amino acid sequence at least 90% identical to SEQ ID NO: 1, and/or
      introducing a nucleic acid into a Solanum spp. plant, plant part, or plant cell, wherein said nucleic acid encodes an amino acid sequence at least 90% identical to SEQ ID NO: 2; and
   b) introducing a nucleic acid into the Solanum spp. plant, plant part, or plant cell, wherein the nucleic acid encodes at least one functional homolog of a glycoalkaloid metabolism (GAME) polypeptide that is a glycotransferase;
      wherein expression of the nucleic acids in (a) and (b) produce tetraose glycoalkaloids, and
      wherein the tetraose glycoalkaloids confer resistance to a fungal pathogen.
16. The method of embodiment 15, wherein the nucleic acid of a) comprises SEQ ID NO: 3, or a sequence at least 75% identical thereto.
17. The method of embodiment 15, wherein the nucleic acid of a) comprises SEQ ID NO: 4, or a sequence at least 75% identical thereto.
18. The method of any one of embodiments 15-17, wherein the glycosyltransferase is a functional homolog of GAME18 and/or GAME17.
19. The method of any one of embodiments 15-18, wherein the nucleic acid in part a) encodes an amino acid sequence at least 90% identical to SEQ ID NO: 1 and the nucleic acid in part b) encodes an amino acid sequence at least 90% identical to SEQ ID NO: 10.
20. The method of any one of embodiments 15-18, wherein the nucleic acid in part a) encodes an amino acid sequence at least 90% identical to SEQ ID NO: 2 and the nucleic acid in part b) encodes an amino acid sequence at least 90% identical to SEQ ID NO: 10.
21. The method of any one of embodiments 15-18, wherein the nucleic acid in part a) encodes an amino acid sequence at least 90% identical to SEQ ID NO: 1 and an amino acid sequence at least 90% identical to SEQ ID NO: 2, and the nucleic acid in part b) encodes an amino acid sequence at least 90% identical to SEQ ID NO: 10.
22. The method of any one of embodiments 15-18, wherein the nucleic acid in part a) encodes an amino acid sequence at least 90% identical to SEQ ID NO: 1 and the nucleic acid in part b) encodes an amino acid sequence at least 90% identical to SEQ ID NO: 12.
23. The method of any one of embodiments 15-18, wherein the nucleic acid in part a) encodes an amino acid sequence at least 90% identical to SEQ ID NO: 2 and the nucleic acid in part b) encodes an amino acid sequence at least 90% identical to SEQ ID NO: 12.
24. The method of any one of embodiments 15-18, wherein the nucleic acid in part a) encodes an amino acid sequence at least 90% identical to SEQ ID NO: 1 and an amino acid sequence at least 90% identical to SEQ ID NO: 2, and the nucleic acid in part b) encodes an amino acid sequence at least 90% identical to SEQ ID NO: 12.
25. The method of any one of embodiments 15-18, wherein the nucleic acid in part a) encodes an amino acid sequence at least 90% identical to SEQ ID NO: 1 and the nucleic acid in part b) encodes an amino acid sequence at least 90% identical to SEQ ID NO: 10 and an amino acid sequence at least 90% identical to SEQ ID NO: 12.
26. The method of any one of embodiments 15-18, wherein the nucleic acid in part a) encodes an amino acid sequence at least 90% identical to SEQ ID NO: 2 and the nucleic acid in part b) encodes an amino acid sequence at least 90% identical to SEQ ID NO: 10 and an amino acid sequence at least 90% identical to SEQ ID NO: 12.
27. The method of any one of embodiments 15-18, wherein the nucleic acid in part a) encodes an amino acid sequence at least 90% identical to SEQ ID NO: 1 and an amino acid sequence at least 90% identical to SEQ ID NO: 2, and the nucleic acid in part b) encodes an amino acid sequence at least 90% identical to SEQ ID NO: 10 and an amino acid sequence at least 90% identical to SEQ ID NO: 12.
28. The method of any one of embodiments 1-13 or 15-27, further comprising introducing an Sgt3 antisense transgene to the Solanum spp. plant, plant part, or plant cell.
29. The method of any one of embodiments 1-13 or 15-27, wherein the Solanum spp. plant, plant part, or plant cell, comprises a non-functional Sgt3 allele.
30. The method of any one of embodiments 1-13 or 15-27, wherein expression of Sgt3 in the Solanum spp. plant, plant part, or plant cell, has been knocked out or reduced.
31. The method of any one of embodiments 15-30, wherein the S. tuberosum plant is resistant to at least one of the following fungal pathogens: Alternaria spp., Colletotrichum coccodes, Rhizoctonia solani Fusarium spp., Phoma spp., Helminthosporium solani, Polyscytalum pustulans, Verticillium spp., Helicobasidium mompa, and Synchytrium endobioticum. Sclerotina spp., Botrytis cinereal.
32. The method of embodiment 31, wherein the fungal pathogen is Alternaria alternata or Alternaria solani.
33. A S. tuberosum plant produced by the method of any one of embodiments 15-32, wherein the plant is resistant Alternaria alternata or Alternaria solani.
34. The S. tuberosum plant of embodiment 33, wherein the plant is S. tuberosum subsp. tuberosum.
35. The S. tuberosum plant of embodiment 33, wherein the plant is a cultivated S. tuberosum selected from the group consisting of Adirondack Blue, Adirondack Red, Agata, Almond, Amandine, Anya, Arran Victory, Atlantic, Bamberg, Belle de Fontenay, BF-15, Bildtstar, Bintje, Blue Congo, Bonnotte, Cabritas, Camota, Chelina, Chiloé, Cielo, Clavela Blanca, Désirée, Fianna, Fingerling, Flava, Golden Wonder, Innovator, Jersey Royal, Kerr's Pink, Kestrel, King Edward, Kipfler, Lady Balfour, Linda, Marfona, Maris Piper, Marquis, Nicola, Pachacoñ, Pink Eye, Pink Fir Apple, Primura, Ratte, Red Norland, Red Pontiac, Rooster, Russet Burbank, Russet Norkotah, Selma, Shepody, Sieglinde, Sirco, Spunta, Stobrawa, Vivaldi, Vitelotte, Yellow Finn, and Yukon Gold.
36. A composition comprising:
an effective amount of isolated tetraose glycoalkaloids; and
an adjuvant
37. The composition of embodiment 36, wherein the tetraose glycoalkaloids are selected from the group consisting of Solanidine-Gal-Glu-Glu-Glu, Solanidine-Gal-Glu-Glu-Xyl, and Solanidine-Gal-Glu-Rha-Glu.
38. The composition of embodiment 36 or 37, wherein the composition is a fungicide.
39. The composition of embodiment 38, wherein the composition is a broad-spectrum fungicide.
40. The composition of any one of embodiments 36-39, further comprising an additional compound selected from the group consisting of azoxystrobin, bifujunzhi, coumethoxystrobin, coumoxystrobin, dimoxystrobin, enestroburin, enoxastrobin, fenaminstrobin, fenoxystrobin, flufenoxystrobin, fluoxastrobin, jiaxiangjunzhi, kresoxim-methyl, mandestrobin, metominostrobin, orysastrobin, picoxystrobin, pyraclostrobin, pyrametostrobin, pyraoxystrobin, triclopyricarb, trifloxystrobin, methyl 2-[2-(2,5-dimethylphenyloxymethyl)phenyl]-3-methoxyacrylate, azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole, imazalil, pefurazoate, prochloraz, triflumizole, pyrimidines, fenarimol, nuarimol, pyrifenox, and triforine.
41. The composition of any one of embodiments 36-40, wherein the adjuvant is a surfactant.
42. The composition of embodiment 41, wherein the surfactant is a non-ionic surfactant, crop oil concentrate, nitrogen-surfactant blend, esterified seed oil, or an organo-silicone.
43. The composition of any one of embodiments 36-42, wherein the composition is a powder, granule, or liquid.
44. A method for controlling fungal and fungal-like pathogens on a plant, comprising applying an effective amount of isolated tetraose glycoalkaloids.
45. The method of embodiment 44, wherein the tetraose glycoalkaloids are selected from the group consisting of Solanidine-Gal-Glu-Glu-Glu, Solanidine-Gal-Glu-Glu-Xyl, and Solanidine-Gal-Glu-Rha-Glu.
46. The method of embodiment 44 or 45, wherein the effective amount of tetraose glycoalkaloids is applied as a dust, granule, gas, or liquid.
47. The method of embodiment 46, wherein the fungal or fungal-like pathogen is Septoria tritici, Mycosphaerella, graminicola, Puccinia spp, Venturia inaequalis, Ustilago maydis, Uncinula necator, Rhynchosporium secalis, Leptosphaeria nodorum, Magnaporthe grisea, Monilinia fructicola, Pseudoperonospora cubensis, Pseudocercosporella herpotrichoides, Phakopsora pachyrhizi, Phaeosphaeria nodorum, Blumoria spp., Ersiphe cichoracearum, Ezysiphe graaminis, Glomerella lagenarluin, Cercospora beticola, Alternaria spp., Rhizoctonia solani, Plasmopara viticola, Phytophthora infestans, Pyricularia oryzae, Pyrenophora teres, Colletotrichum coccodes, Rhizoctonia solani Fusarium spp., Phoma spp., Helminthosporium solani, Polyscytalum pustulans, Verticillium spp., Helicobasidium mompa, and Synchytrium endobioticum.
48. The method of any one of embodiments 44-47, wherein the plant is selected from the group consisting of: wheat, pear, apple, corn, grape, barley, rye, rice, peach, melon, cucumber, pumpkin, squash, sorghum, millet, soybean, sugar beet, spinach, swiss chard, potato, eggplant, tomato, strawberry, and grapevine.
49. A plant transformation vector designated pSIMScGTR1 comprising the nucleotide sequence set forth in SEQ ID NO: 3, and wherein the plant transformation vector was deposited under ATCC Accession No. PTA-______.
50. A plant transformation vector designated pSIMScGTR2 comprising the nucleotide sequence set forth in SEQ ID NO: 4, and wherein the plant transformation vector was deposited under ATCC Accession No. PTA-______.
51. A plant transformation vector designated pSIML2GGA1 comprising the nucleotide sequences set forth in SEQ ID NOs: 3 and 9, and wherein the plant transformation vector was deposited under ATCC Accession No. PTA-______.
52. A plant transformation vector designated pSIML2GGA2 comprising the nucleotide sequences set forth in SEQ ID NOs: 4 and 9, and wherein the plant transformation vector was deposited under ATCC Accession No. PTA-______.
53. A plant transformation vector designated pSIML2GGA3 comprising the nucleotide sequences set forth in SEQ ID NOs: 3, 4, and 9, and wherein the plant transformation vector was deposited under ATCC Accession No. PTA-_______.
54. A plant transformation vector comprising at least one of:
   a nucleotide sequence encoding an amino acid sequence at least 90% identical to SEQ ID NO: 1;
   a nucleotide sequence encoding an amino acid sequence at least 90% identical to SEQ ID NO: 2;
   a nucleotide sequence encoding an amino acid sequence at least 90% identical to SEQ ID NO: 10; and
   a nucleotide sequence encoding an amino acid sequence at least 90% identical to SEQ ID NO: 12.
55. A plant transformed with the vector of any one of embodiments 49-54.
56. The plant of embodiment 55, wherein the plant is resistant to a fungal pathogen.
57. A Solanum tuberosum plant, plant part, or plant cell, comprising a nucleic acid encoding an amino acid sequence at least 90% identical to SEQ ID NO: 1.
58. The Solanum tuberosum plant, plant part, or plant cell, of embodiment 57, wherein the nucleic acid is SEQ ID NO: 3, or a sequence at least 75% identical thereto.
59. A Solanum tuberosum plant, plant part, or plant cell, comprising a nucleic acid encoding an amino acid sequence at least 90% identical to SEQ ID NO: 2.
60. The Solanum tuberosum plant, plant part, or plant cell, of embodiment 59, wherein the nucleic acid is SEQ ID NO: 4, or a sequence at least 75% identical thereto.
61. A Solanum tuberosum plant, plant part, or plant cell, comprising a nucleic acid encoding an amino acid sequence at least 90% identical to SEQ ID NO: 10.
62. The Solanum tuberosum plant, plant part, or plant cell, of embodiment 61, wherein the nucleic acid is SEQ ID NO: 9, or a sequence at least 75% identical thereto.
63. A Solanum tuberosum plant, plant part, or plant cell, comprising a nucleic acid encoding an amino acid sequence at least 90% identical to SEQ ID NO: 12.
64. The Solanum tuberosum plant, plant part, or plant cell, of embodiment 63, wherein the nucleic acid is SEQ ID NO: 11, or a sequence at least 75% identical thereto.
65. The Solanum tuberosum plant, plant part, or plant cell of any one of embodiments 57-64, wherein the plant is resistant to a fungal pathogen.
66. The Solanum tuberosum plant, plant part, or plant cell of any one of embodiments 57-65, wherein the plant is S. tuberosum subsp. tuberosum.
67. A sequence fragment of at least 15 consecutive nucleotides of the ScGTR1 gene described by SEQ ID NO: 3 or its complementary sequence.
68. The fragment of embodiment 67, wherein said fragment is isolated.
69. A method for identifying a plant comprising a ScGTR1 allele, comprising using the fragment of embodiment 67 or 68.
70. A sequence fragment of at least 15 consecutive nucleotides of the ScGTR2 gene described by SEQ ID NO: 4 or its complementary sequence.
71. The fragment of embodiment 70, wherein said fragment is isolated.
72. A method for identifying a plant comprising a ScGTR2 allele, comprising using the fragment of embodiment 70 or 71.
73. A sequence fragment of at least 15 consecutive nucleotides of the ScGAME18 gene described by SEQ ID NO: 9 or its complementary sequence.
74. The fragment of embodiment 73, wherein said fragment is isolated.
75. A method for identifying a plant comprising a ScGAME18 allele, comprising using the fragment of embodiment 73 or 74.
76. A sequence fragment of at least 15 consecutive nucleotides of the ScGAME17 gene described by SEQ ID NO: 11 or its complementary sequence.
77. The fragment of embodiment 76, wherein said fragment is isolated.
78. A method for identifying a plant comprising a ScGAME17 allele, comprising using the fragment of embodiment 76 or 77.

What is claimed is:

1. A method for conferring resistance or tolerance to a pest or pathogen in a Solanum spp. plant, plant part, or plant cell, comprising at least one of:
a) introducing a nucleic acid into a Solanum spp. plant, plant part, or plant cell, wherein said nucleic acid encodes an amino acid sequence at least 90% identical to SEQ ID NO: 1, and
b) introducing a nucleic acid into a Solanum spp. plant, plant part, or plant cell, wherein said nucleic acid encodes an amino acid sequence at least 90% identical to SEQ ID NO: 2.

2. The method of claim 1, wherein the nucleic acid of a) comprises SEQ ID NO: 3, or a sequence at least 75% identical thereto, and wherein the nucleic acid of b) comprises SEQ ID NO: 4, or a sequence at least 75% identical thereto.

3. The method of claim 1, further comprising introducing a nucleic acid encoding a functional homolog of GAME18 and/or GAME17.

4. The method of claim 3, wherein the nucleic acid encodes an amino acid sequence at least 90% identical to SEQ ID NO: 10 and/or an amino acid sequence at least 90% identical to SEQ ID NO: 12.

5. The method of claim 1, wherein expression of at least one of the nucleic acids generates tetraose glycoalkaloids, and wherein the tetraose glycoalkaloids confers resistance to a pest or pathogen.

6. The method of claim 5, wherein the Solanum spp. plant is resistant to at least one of the following fungal pathogens: Alternaria spp., Colletotrichum coccodes, Rhizoctonia solani Fusarium spp., Phoma spp., Helminthosporium solani, Polyscytalum pustulans, Verticillium spp., Helicobasidium mompa, and Synchytrium endobioticum. Botrytis cinerea, Sclerotina spp, Septoria spp.

7. The method of claim 3, further comprising introducing an Sgt3 antisense transgene to the Solanum spp. plant, plant part, or plant cell.

8. The method of claim 3, wherein the Solanum spp. plant, plant part, or plant cell, comprises a non-functional Sgt3 allele.

9. The method of claim 3, wherein expression of Sgt3 in the Solanum spp. plant, plant part, or plant cell, has been knocked out or reduced.

10. The method of claim 1, wherein the Solanum spp. plant, plant part, or plant cell is S. tuberosum, S. melongena, or S. lycopersicum.

11. A Solanum spp. plant produced by the method of claim 1.

12. A S. tuberosum plant produced by the method of claim 3, wherein the plant is resistant Alternaria alternata or Alternaria solani.

13. The S. tuberosum plant of claim 12, wherein the plant is S. tuberosum subsp. tuberosum.

14. The S. tuberosum plant of claim 12, wherein the plant is a cultivated S. tuberosum selected from the group consisting of Adirondack Blue, Adirondack Red, Agata, Almond, Amandine, Anya, Arran Victory, Atlantic, Bamberg, Belle de Fontenay, BF-15, Bildtstar, Bintje, Blue Congo, Bonnotte, Cabritas, Camota, Chelina, Chiloé, Cielo, Clavela Blanca, Désirée, Fianna, Fingerling, Flava, Golden Wonder, Innovator, Jersey Royal, Kerr's Pink, Kestrel, King Edward, Kipfler, Lady Balfour, Linda, Marfona, Maris Piper, Marquis, Nicola, Pachacoña, Pink Eye, Pink Fir Apple, Primura, Ratte, Red Norland, Red Pontiac, Rooster, Russet Burbank, Russet Norkotah, Selma, Shepody, Sieglinde, Sirco, Spunta, Stobrawa, Vivaldi, Vitelotte, Yellow Finn, and Yukon Gold.

15. A plant transformation vector comprising at least one of:
a nucleotide sequence encoding an amino acid sequence at least 90% identical to SEQ ID NO: 1;
a nucleotide sequence encoding an amino acid sequence at least 90% identical to SEQ ID NO: 2;
a nucleotide sequence encoding an amino acid sequence at least 90% identical to SEQ ID NO: 10; and
a nucleotide sequence encoding an amino acid sequence at least 90% identical to SEQ ID NO: 12.

16. A plant transformed with the vector of claim 15.

17. The plant of claim 16, wherein the plant is resistant to a fungal pathogen.

18. A Solanum tuberosum plant, plant part, or plant cell, comprising at least one of:
a nucleic acid encoding an amino acid sequence at least 90% identical to SEQ ID NO: 1, or a nucleic acid sequence at least 75% identical to SEQ ID NO: 3;
a nucleic acid encoding an amino acid sequence at least 90% identical to SEQ ID NO: 2, or a nucleic acid sequence at least 75% identical to SEQ ID NO: 4;
a nucleic acid encoding an amino acid sequence at least 90% identical to SEQ ID NO: 10, or a nucleic acid sequence at least 75% identical to SEQ ID NO: 9; and
a nucleic acid encoding an amino acid sequence at least 90% identical to SEQ ID NO: 12, or a nucleic acid sequence at least 75% identical to SEQ ID NO: 11.

19. The Solanum tuberosum plant of claim 18, wherein the plant further comprises an Sgt3 antisense transgene.

20. The Solanum tuberosum plant, plant part, or plant cell of claim 18, wherein the plant is resistant to a fungal pathogen.
